(19) United States
(12) Patent Application Publication
(10) Pub. No.: US 2019/0337921 A1
(43) Pub. Date: Nov. 7, 2019

(54) TETRASUBSTITUTED ALKENE COMPOUNDS AND THEIR USE

(71) Applicant: Eisai R&D Management Co., Ltd., Tokyo (JP)

(72) Inventors: Mark Bock, Boston, MA (US); Ming-Hong Hao, Quincy, MA (US); Manav Korpal, Winchester, MA (US); Vijay Kumar Nyavanandi, Andhra Pradesh (IN); Xiaoling Puyang, Cambridge, MA (US); Susanta Samajdar, Karnataka (IN); Peter Gerard Smith, Arlington, MA (US); John Wang, Andover, MA (US); Guo Zhu Zheng, Lexington, MA (US); Ping Zhu, Acton, MA (US); Lorna Helen Mitchell, Cambridge, MA (US); Nicholas Larsen, Needham, MA (US); Nathalie Rioux, Woburn, MA (US)

(73) Assignee: Eisai R&D Management Co., Ltd., Tokyo (JP)

(21) Appl. No.: 16/349,508

(22) PCT Filed: Nov. 22, 2017
(86) PCT No.: PCT/US2017/062978

§ 371 Date: May 13, 2019

(30) Foreign Application Priority Data
Nov. 24, 2016 (IN) 201641040208

(51) Int. Cl. C07D 401/06; C07D 209/08; C07D 401/14; A61P 35/00

(52) U.S. Cl. CPC C07D 401/06; A61P 35/00; C07D 401/14; C07D 209/08

(57) ABSTRACT

Disclosed herein are compounds, or pharmaceutically acceptable salts thereof, and methods of using the compounds for treating breast cancer by administration to a subject in need thereof a therapeutically effective amount of the compounds or pharmaceutically acceptable salts thereof. The breast cancer may be an ER-positive breast cancer and/or the subject in need of treatment may express a mutant ER-α protein.

### CROSS-REFERENCE TO RELATED APPLICATIONS

This application claims the benefit of priority of Indian Provisional Patent Application No. 201641040208, filed on Nov. 24, 2016. That application is incorporated by reference as if fully rewritten herein.

### BACKGROUND

Breast cancer is the most commonly diagnosed malignancy among women today with nearly 200,000/1.7 million new cases diagnosed in the US/worldwide each year respectively. Since about 70% of breast tumors are positive for the estrogen receptor alpha (ERα)—a key oncogenic driver in this subset of tumors—several classes of therapies have been developed to antagonize ERα function, including 1) selective estrogen receptor downregulators (SERDs) of which fulvestrant is an example, 2) selective estrogen receptor modulators (SERMs) of which tamoxifen is an example and 3) aromatase inhibitors that reduce systemic levels of estrogen. These therapies have been largely effective in the clinic reducing occurrence and progression of ERα+ breast tumors. However there are on-target liabilities associated with these different classes of compounds. For example, tamoxifen has been shown to activate signaling activity in the endometrium leading to an increase in risk of endometrial cancers in the clinic (Fisher et al., (1994) J Natl Cancer Inst. April 6; 86(7):527-37; van Leeuwen et al., (1994) Lancet February 19; 343(8895):448-52). In contrast, since fulvestrant is a pure antagonist, it can lead to loss of bone density in post-menopausal women as ERα activity is critical for bone building. In addition to on-target side effects, clinical resistance is also beginning to emerge to these classes of ERα antagonists highlighting the need to develop next-generation compounds.

Several mechanisms of resistance have been identified using in vitro and in vivo models of resistance to various endocrine therapies. These include increased ERα/HER2 “crosstalk” (Shou et al., (2004) J Natl Cancer Inst. June 16; 96(12):926-35), aberrant expression of ERα coactivators/corepressors (Osborne et al., (2003) J Natl Cancer Inst. March 5; 95(5):353-61) or loss of ERα altogether to allow ER-independent growth (Osborne C K, Schiff R (2011) Annu Rev Med 62: 233-47).

In the hopes of identifying clinically relevant mechanisms of resistance, great effort has also recently gone into deeply characterizing the genetics of endocrine-therapy resistant metastases isolated from patients. Several independent labs have recently published the multitude of genetic lesions observed in the resistant vs the primary tumors (Li et al., (2013) Cell Rep. September 26; 4(6): 1116-30; Robinson et al., (2013) Nat Genet. December; 45(12): 1446-51; Toy et al., (2013) Nat Genet. 2013 December; 45(12): 1439-45). Among these are the highly recurrent mutations in the ligand-binding domain of ESR1 (gene which encodes ERα protein) found to be significantly enriched in about 20% of resistant tumors relative to endocrine therapy naïve tumors (Jeselsohn et al., (2014) Clin Cancer Res. April 1; 20(7): 1757-67; Toy et al., (2013) Nat Genet. 2013 December; 45(12):1439-45; Robinson et al., (2013) Nat Genet. December; 45(12):1446-51; Merenbakh-Lamin et al., (2013) Cancer Res. December 1; 73(23):6856-64; Yu et al., (2014) Science July 11; 345(6193):216-20; Segal and Dowsett (2014), Clin Cancer Res April 1; 20(7):1724-6), suggesting the potential for these mutations to functionally drive clinical resistance. In contrast to the enrichment in ESR1 mutations observed in therapy-resistant tumors, mutations in other cancer-related genes failed to show such a robust enrichment strongly implying the importance of ERα mutations in promoting resistance (Jeselsohn et al., (2014) Clin Cancer Res. April 1; 20(7):1757-67).

ER+ breast cancer patients on average are treated with seven independent therapies including chemotherapies and various anti-estrogen therapies such as tamoxifen, fulvestrant and aromatase inhibitors. Recent genomic profiling has revealed that the ERα pathway remains a critical driver of tumor growth in the resistant setting as activating mutations in ERα have emerged. Thus, it is critical that more potent ER-directed therapies be developed that can overcome resistance in the clinical setting. Hence, there is a need for novel compounds that can potently suppress the growth of both wild-type (WT) and ER α-mutant positive tumors.

### SUMMARY

Described herein are novel compounds useful for treating cancer.

Embodiments may provide a compound given by Formula (X):

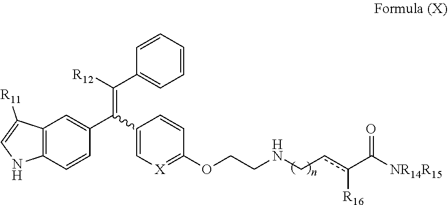

wherein:

R₁₁ is —H or —F;

R₁₂ is —CH₂CH₃, —CH₂CF₃, or cyclobutyl;

R₁₄ and R₁₅
i) are the same or different and are independently selected from —H, —CH₃, and —CH₂CH₂OH; or
ii) form a 4-6 membered heterocycloalkyl ring with the N to which they are attached, optionally with an additional heteroatom in the 4-6 membered ring;

R₁₆ is —H or forms a 5-7 membered heterocycloalkyl ring with R₁₄ and the N to which R₁₄ is attached;

X is N or C;

n is 1-2;

[symbol] represents a single bond or a double bond;

wherein R₁₆ is H when R₁₄ and R₁₅ form said 4-6 membered heterocycloalkyl ring; and

wherein R₁₅ is selected from —H, —CH₃, and —CH₂CH₂OH when R₁₆ forms said 5-7 membered heterocycloalkyl ring with R₁₄; or a pharmaceutically acceptable salt thereof.

A further embodiment may provide a compound with the following stereochemistry:

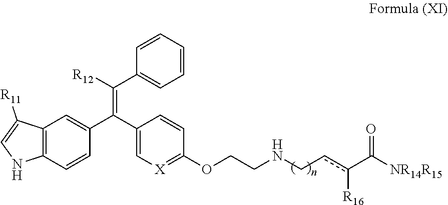

or a pharmaceutically acceptable salt thereof.

A further embodiment provides a compound or pharmaceutically acceptable salt as shown above wherein R₁₁ is —F. A still further embodiment provides a compound or pharmaceutically acceptable salt as shown above wherein R₁₁ is —H. A yet still further embodiment provides a compound or pharmaceutically acceptables salt as shown above wherein R₁₂ is —CH₂—CF₃. Another embodiment provides a compound or pharmaceutically acceptable salt as shown above wherein R₁₂ is —CH₂CH₃. In a further embodiment X is N. A further embodiment shows a compound or pharmaceutically acceptable salt as shown above, wherein R₁₄ is H and R₁₅ is —CH₃. A still further embodiment shows a compound or pharmaceutically acceptable salt as shown above wherein R₁₄ and R₁₅ are —CH₃. Another embodiment shows a compound or pharmaceutically acceptable salt as shown above wherein [symbol] represents a double bond.

Further embodiments may include a compound selected from the group consisting of:
(2E)-N,N-dimethyl-4-[[2-([5-[(1E)-4,4,4-trifluoro-1-(1H-indol-5-yl)-1-phenylbut-1-en-2-yl]pyridin-2-yl]oxy)ethyl]amino]but-2-enamide;
(E)-4-(2-(4-((E)-1-(1H-indol-5-yl)-2-phenylbut-1-enyl)phenoxy)ethyl amino)-N,N-dimethylbut-2-enamide;
(E)-N,N-dimethyl-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-enamide;
(E)-N-methyl-4-((2-((5-((Z)-4,4,4-trifluoro-1-(1H-indol-5-yl)-2-phenylbut-1-en-1-yl)pyridin-2-yl)oxy)ethyl)amino)but-2-enamide;
(E)-N-methyl-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-enamide;
(E)-N-methyl-4-((2-(4-((E)-4,4,4-trifluoro-1-(1H-indol-5-yl)-2-phenylbut-1-en-1-yl)phenoxy)ethyl)amino)but-2-enamide;
(E)-N-methyl-4-((2-(4-((E)-4,4,4-trifluoro-1-(3-fluoro-1H-indol-5-yl)-2-phenylbut-1-en-1-yl)phenoxy)ethyl)amino)but-2-enamide;
(E)-1-(pyrrolidin-1-yl)-4-((2-((5-((Z)-4,4,4-trifluoro-1-(1H-indol-5-yl)-2-phenylbut-1-en-1-yl)pyridin-2-yl)oxy)ethyl)amino)but-2-en-1-one;
(E)-1-(pyrrolidin-1-yl)-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-en-1-one;
(E)-4-((2-((5-((Z)-2-cyclobutyl-1-(1H-indol-5-yl)-2-phenylvinyl)pyridin-2-yl)oxy)ethyl)amino)-N-methylbut-2-enamide;
(E)-N-(2-hydroxyethyl)-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-enamide;
(E)-N-(2-hydroxyethyl)-4-((2-((5-((Z)-4,4,4-trifluoro-1-(1H-indol-5-yl)-2-phenylbut-1-en-1-yl)pyridin-2-yl)oxy)ethyl)amino)but-2-enamide;
(E)-N-(2-hydroxyethyl)-5-((2-((5-((Z)-4,4,4-trifluoro-1-(3-fluoro-1H-indol-5-yl)-2-phenylbut-1-en-1-yl)pyridin-2-yl)oxy)ethyl)amino)pent-2-enamide;
(Z)—N-methyl-4-((2-((5-(4,4,4-trifluoro-1-(3-fluoro-1H-indol-5-yl)-2-phenylbut-1-en-1-yl)pyridin-2-yl)oxy)ethyl)amino)butanamide;
(Z)—N-methyl-4-((2-((5-(4,4,4-trifluoro-1-(1H-indol-5-yl)-2-phenylbut-1-en-1-yl)pyridin-2-yl)oxy)ethyl)amino)butanamide;
(Z)—N-methyl-5-((2-((5-(4,4,4-trifluoro-1-(3-fluoro-1H-indol-5-yl)-2-phenylbut-1-en-1-yl)pyridin-2-yl)oxy)ethyl)amino)pentanamide;
(Z)—N-methyl-5-((2-((5-(4,4,4-trifluoro-1-(1H-indol-5-yl)-2-phenylbut-1-en-1-yl)pyridin-2-yl)oxy)ethyl)amino)pentanamide;
(Z)-3-(2-((2-((5-(4,4,4-trifluoro-1-(3-fluoro-1H-indol-5-yl)-2-phenylbut-1-en-1-yl)pyridin-2-yl)oxy)ethyl)amino)ethyl)pyrrolidin-2-one;
(Z)-3-(2-((2-((5-(4,4,4-trifluoro-1-(1H-indol-5-yl)-2-phenylbut-1-en-1-yl)pyridin-2-yl)oxy)ethyl)amino)ethyl)pyrrolidin-2-one;
(E)-4-((2-((5-((Z)-1-(3-fluoro-1H-indol-5-yl)-2-phenylbut-1-en-1-yl)pyridin-2-yl)oxy)ethyl)amino)-N-methylbut-2-enamide;
(E)-4-((2-(4-((E)-1-(3-fluoro-1H-indol-5-yl)-2-phenylbut-1-en-1-yl)phenoxy)ethyl)amino)-N-methylbut-2-enamide;
(E)-4-((2-(4-((E)-2-cyclobutyl-1-(3-fluoro-1H-indol-5-yl)-2-phenylvinyl)phenoxy)ethyl)amino)-N-methylbut-2-enamide;
(E)-4-((2-((5-((Z)-2-cyclobutyl-1-(3-fluoro-1H-indol-5-yl)-2-phenylvinyl)pyridin-2-yl)oxy)ethyl)amino)-N-methylbut-2-enamide; and
(E)-4-((2-((5-((Z)-2-cyclobutyl-1-(1H-indol-5-yl)-2-phenylvinyl)pyridin-2-yl)oxy)ethyl)amino)-N-methylbut-2-enamide; or a pharmaceutically acceptable salt thereof.

A further embodiment provides compound having the following formula:

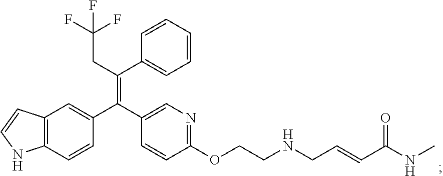 [SMILES: CNC(=O)/C=C/CNCCOc1ccc(/C(=C(/CC(F)(F)F)c2ccccc2)c2ccc3[nH]ccc3c2)cn1]

or a pharmaceutically acceptable salt thereof.

A further embodiment provides a compound having the following formula:

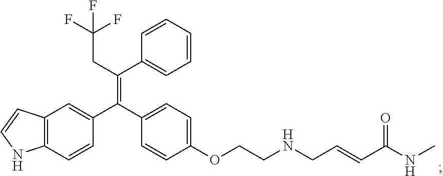 [SMILES: CNC(=O)/C=C/CNCCOc1ccc(/C(=C(/CC(F)(F)F)c2ccccc2)c2ccc3[nH]ccc3c2)cc1]

or a pharmaceutically acceptable salt thereof.

A further embodiment provides a compound of Formula (XII) or a pharmaceutically acceptable salt thereof:

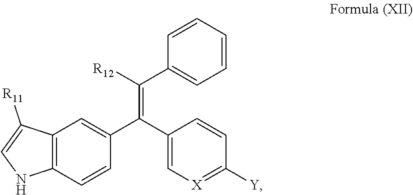

wherein R₁₁ is —H or —F;

R₁₂ is —CH₂CH₃, —CH₂CF₃, or cyclobutyl;

X is N or C; and

Y is one of the following:

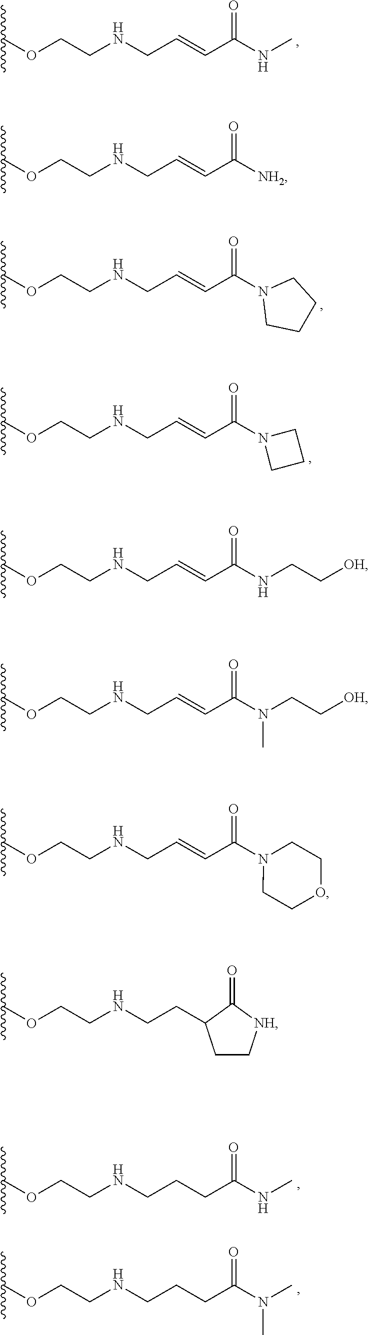

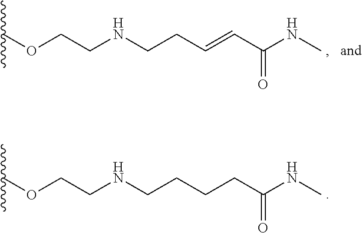 [SMILES: *OCCNCC/C=C/C(=O)NC ; *OCCNCCCCC(=O)NC]

A further embodiment may provide a method of treating breast cancer comprising administering to a subject a compound or pharmaceutically acceptable salt according to any one of the preceding paragraphs. The breast cancer may be an ER-positive breast cancer. The subject may express a mutant ER-α protein. An embodiment may provide use of a compound or pharmaceutically acceptable salt as in the paragraphs above for treating breast cancer. In some embodiments the breast cancer is an ER-positive breast cancer. In some embodiments said subject expresses a mutant ER-α protein. In some embodiments a compound or pharmaceutically acceptable salt as presented above is used in the preparation of a medicament for treatment of breast cancer.

In embodiments, the compounds disclosed herein are useful for inhibiting the cell culture growth of MCF7 ER-alpha (wildtype) and MCF7 ER-alpha (Y537S mutant) cells. Other compounds (e.g., tamoxifen, raloxifene and fulvestrant) known to inhibit the cell culture growth of MCF7 ER-alpha (wildtype) cells are currently used to treat breast cancer in human patients. Hence, the compounds disclosed herein are useful for treating ER-alpha expressing breast cancer in human patients, and are useful for treating Y537S mutant ER-alpha expressing breast cancer in human patients.

In embodiments, the compounds disclosed herein are useful for treating breast cancer. In embodiments, the breast cancer is ER-α+. In embodiments, the breast cancer expresses an ER-α mutation, which is L536Q (Robinson et al. Nat Genet. 2013 December; 45(12)), L536R (Toy et al. Nat Genet. 2013 December; 45(12):1439-45), Y537S (Toy et al. Nat Genet. 2013 December; 45(12): 1439-45: Robinson et al. Nat Genet. 2013 December; 45(12); Jeselsohn et al. Clin Cancer Res. 2014 Apr. 1; 20(7): 1757-67), Y537N (Toy et al. Nat Genet. 2013 December; 45(12): 1439-45; Jeselsohn et al. Clin Cancer Res. 2014 Apr. 1; 20(7): 1757-67), Y537C (Toy et al. Nat Genet. 2013 December; 45(12): 1439-45: Jeselsohn et al. Clin Cancer Res. 2014 Apr. 1; 20(7):1757-67) and D538G (Toy et al. Nat Genet. 2013 December; 45(12):1439-45; Robinson et al. Nat Genet. 2013 December; 45(12); Jeselsohn et al. Clin Cancer Res. 2014 Apr. 1; 20(7):1757-67; Merenbakh-Lamin et al. Cancer Res. 2013 Dec. 1; 73(23):6856-64); and Yu et al., (2014) Science July 11; 345(6193):216-20, all of which are incorporated by reference in their entireties for their teachings of ER-α mutations.

### BRIEF DESCRIPTION OF THE DRAWINGS

FIG. 1 shows in vitro proliferation effects of wild-type and mutant ER-bearing MCF7 lines to clinical therapies 4-hydroxytamoxifen (4-OHT), raloxifene and fulvestrant, where phenotypic resistance observed in mutant-bearing lines relative to control lines to existing clinical compounds, whereby MCF7 cells engineered to overexpress various ERα^{MUT} showed partial resistance to various endocrine therapies.

### DETAILED DESCRIPTION

Embodiments may provide a compound given by Formula (X):

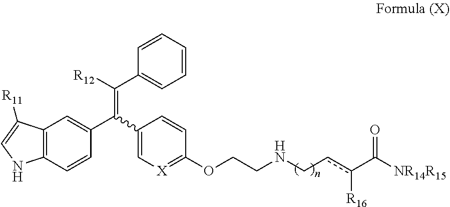

wherein:

R₁₁ is —H or —F;

R₁₂ is —CH₂CH₃, —CH₂CF₃, or cyclobutyl;

R₁₄ and R₁₅
i) are the same or different and are independently selected from H, —CH₃, and —CH₂CH₂OH; or
ii) form a 4-6 membered heterocycloalkyl ring with the N to which they are attached, optionally with an additional heteroatom in the 4-6 membered ring;

R₁₆ is H or forms a 5-7 membered heterocycloalkyl ring with R₁₄ and the N to which R₁₄ is attached;

X is N or C;

n is 1-2;

[symbol] represents a single bond or a double bond;

wherein R₁₆ is H when R₁₄ and R₁₅ form said 4-6 membered heterocycloalkyl ring; and

wherein R₁₅ is selected from H, —CH₃, and —CH₂CH₂OH when R₁₆ forms said 5-7 membered heterocycloalkyl ring with R₁₄; or a pharmaceutically acceptable salt thereof.

A further embodiment may provide a compound with the following stereochemistry:

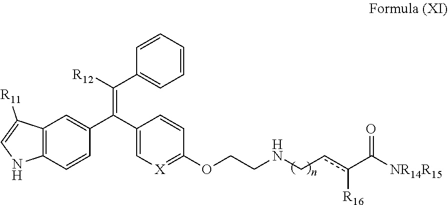

or a pharmaceutically acceptable salt thereof.

A further embodiment provides a compound or pharmaceutically acceptable salt as shown above wherein R₁₁ is —F. A still further embodiment provides a compound or pharmaceutically acceptable salt as shown above wherein R₁₁ is —H. A yet still further embodiment provides a compound or pharmaceutically acceptables salt as shown above wherein R₁₂ is —CH₂—CF₃. Another embodiment provides a compound or pharmaceutically acceptable salt as shown above wherein R₁₂ is —CH₂CH₃. In a further embodiment, X is N. A further embodiment shows a compound or pharmaceutically acceptable salt as shown above, wherein R₁₄ is H and R₁₅ is —CH₃. A still further embodiment shows a compound or pharmaceutically acceptable salt as shown above wherein R₁₄ and R₁₅ are —CH₃. Another embodiment shows a compound or pharmaceutically acceptable salt as shown above wherein [symbol] represents a double bond.

Further embodiments may include a compound selected from the group consisting of:
(2E)-N,N-dimethyl-4-[[2-([5-[(1E)-4,4,4-trifluoro-1-(1H-indol-5-yl)-1-phenylbut-1-en-2-yl]pyridin-2-yl]oxy)ethyl]amino]but-2-enamide;
(E)-4-(2-(4-((E)-1-(1H-indol-5-yl)-2-phenylbut-1-enyl)phenoxy)ethyl amino)-N,N-dimethylbut-2-enamide;
(E)-N,N-dimethyl-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-enamide;
(E)-N-methyl-4-((2-((5-((2-((5-((Z)-4,4,4-trifluoro-1-(1H-indol-5-yl)-2-phenylbut-1-en-1-yl)pyridin-2-yl)oxy)ethyl)amino)but-2-enamide;
(E)-N-methyl-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-enamide;
(E)-N-methyl-4-((2-(4-((E)-4,4,4-trifluoro-1-(1H-indol-5-yl)-2-phenylbut-1-en-1-yl)phenoxy)ethyl)amino)but-2-enamide;
(E)-N-methyl-4-((2-(4-((E)-4,4,4-trifluoro-1-(3-fluoro-1H-indol-5-yl)-2-phenylbut-1-en-1-yl)phenoxy)ethyl)amino)but-2-enamide;
(E)-1-(pyrrolidin-1-yl)-4-((2-((5-((Z)-4,4,4-trifluoro-1-(1H-indol-5-yl)-2-phenylbut-1-en-1-yl)pyridin-2-yl)oxy)ethyl)amino)but-2-en-1-one;
(E)-1-(pyrrolidin-1-yl)-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-en-1-one;
(E)-4-((2-((5-((Z)-2-cyclobutyl-1-(1H-indol-5-yl)-2-phenylvinyl)pyridin-2-yl)oxy)ethyl)amino)-N-methylbut-2-enamide;
(E)-N-(2-hydroxyethyl)-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-enamide;
(E)-N-(2-hydroxyethyl)-4-((2-((5-((Z)-4,4,4-trifluoro-1-(1H-indol-5-yl)-2-phenylbut-1-en-1-yl)pyridin-2-yl)oxy)ethyl)amino)but-2-enamide;
(E)-N-(2-hydroxyethyl)-5-((2-((5-((Z)-4,4,4-trifluoro-1-(3-fluoro-1H-indol-5-yl)-2-phenylbut-1-en-1-yl)pyridin-2-yl)oxy)ethyl)amino)pent-2-enamide;
(Z)—N-methyl-4-((2-((5-(4,4,4-trifluoro-1-(3-fluoro-1H-indol-5-yl)-2-phenylbut-1-en-1-yl)pyridin-2-yl)oxy)ethyl)amino)butanamide;
(Z)—N-methyl-4-((2-((5-(4,4,4-trifluoro-1-(1H-indol-5-yl)-2-phenylbut-1-en-1-yl)pyridin-2-yl)oxy)ethyl)amino)butanamide;
(Z)—N-methyl-5-((2-((5-(4,4,4-trifluoro-1-(3-fluoro-1H-indol-5-yl)-2-phenylbut-1-en-1-yl)pyridin-2-yl)oxy)ethyl)amino)pentanamide;
(Z)—N-methyl-5-((2-((5-(4,4,4-trifluoro-1-(1H-indol-5-yl)-2-phenylbut-1-en-1-yl)pyridin-2-yl)oxy)ethyl)amino)pentanamide;
(Z)-3-(2-((2-((5-(4,4,4-trifluoro-1-(3-fluoro-1H-indol-5-yl)-2-phenylbut-1-en-1-yl)pyridin-2-yl)oxy)ethyl)amino)ethyl)pyrrolidin-2-one;
(Z)-3-(2-((2-((5-(4,4,4-trifluoro-1-(1H-indol-5-yl)-2-phenylbut-1-en-1-yl)pyridin-2-yl)oxy)ethyl)amino)ethyl)pyrrolidin-2-one;
(E)-4-((2-((5-((Z)-1-(3-fluoro-1H-indol-5-yl)-2-phenylbut-1-en-1-yl)pyridin-2-yl)oxy)ethyl)amino)-N-methylbut-2-enamide;
(E)-4-((2-(4-((E)-1-(3-fluoro-1H-indol-5-yl)-2-phenylbut-1-en-1-yl)phenoxy)ethyl)amino)-N-methylbut-2-enamide;
(E)-4-((2-(4-((E)-2-cyclobutyl-1-(3-fluoro-1H-indol-5-yl)-2-phenylvinyl)phenoxy)ethyl)amino)-N-methylbut-2-enamide;
(E)-4-((2-((5-((Z)-2-cyclobutyl-1-(3-fluoro-1H-indol-5-yl)-2-phenylvinyl)pyridin-2-yl)oxy)ethyl)amino)-N-methylbut-2-enamide; and
(E)-4-((2-((5-((Z)-2-cyclobutyl-1-(1H-indol-5-yl)-2-phenylvinyl)pyridin-2-yl)oxy)ethyl)amino)-N-methylbut-2-enamide; or a pharmaceutically acceptable salt thereof.

A further embodiment provides compound having the following formula:

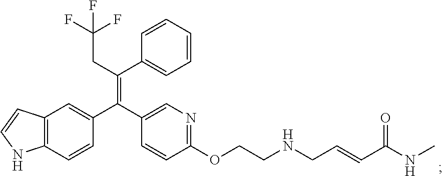 [SMILES: CNC(=O)/C=C/CNCCOc1ccc(/C(=C(/CC(F)(F)F)c2ccccc2)c2ccc3[nH]ccc3c2)cn1]

or a pharmaceutically acceptable salt thereof.

A further embodiment provides a compound having the following formula:

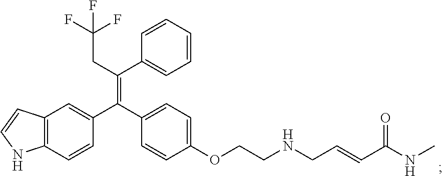 [SMILES: CNC(=O)/C=C/CNCCOc1ccc(/C(=C(/CC(F)(F)F)c2ccccc2)c2ccc3[nH]ccc3c2)cc1]

or a pharmaceutically acceptable salt thereof.

A further embodiment provides a compound of Formula (XII) or a pharmaceutically acceptable salt thereof:

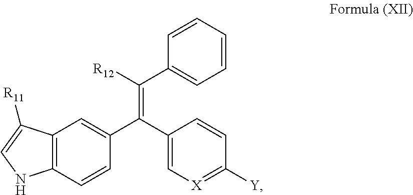

wherein R₁₁ is —H or —F;

R₁₂ is —CH₂CH₃, —CH₂CF₃, or cyclobutyl;

X is N or C; and

Y is one of the following:

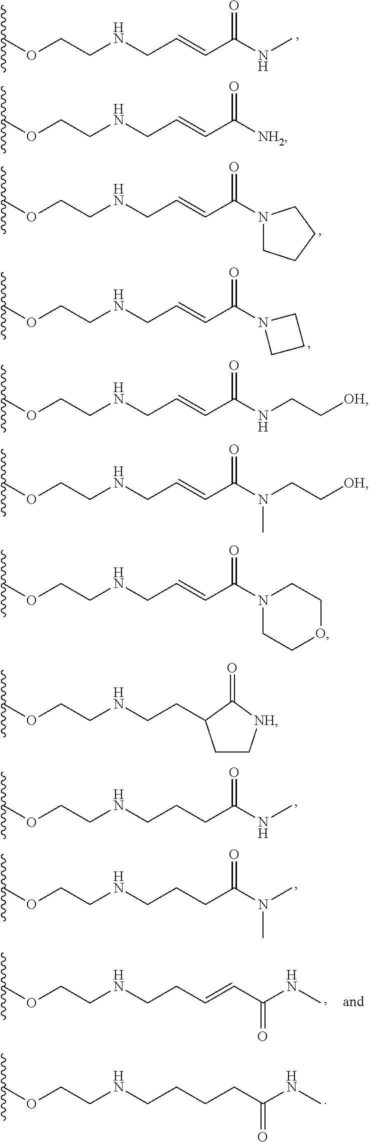

A further embodiment may provide a method of treating breast cancer comprising administering to a subject a compound or pharmaceutically acceptable salt according to any one of the preceding paragraphs. The breast cancer may be an ER-positive breast cancer. The subject may express a mutant ER-α protein. An embodiment may provide use of a compound or pharmaceutically acceptable salt as in the paragraphs above for treating breast cancer. In some embodiments the breast cancer is an ER-positive breast cancer. In some embodiments said subject expresses a mutant ER-α protein. In some embodiments a compound or pharmaceutically acceptable salt as presented above is used in the preparation of a medicament for treatment of breast cancer.

A further embodiment may provide a method of treating breast cancer comprising administering to a subject a compound or pharmaceutically acceptable salt according to any one of the preceding paragraphs. The breast cancer may be an ER-positive breast cancer. The subject may express a mutant ER-α protein. An embodiment may provide use of a compound or pharmaceutically acceptable salt as in the paragraphs above for treating breast cancer. In some embodiments the breast cancer is an ER-positive breast cancer. In some embodiments said subject expresses a mutant ER-α protein. In some embodiments a compound or pharmaceutically acceptable salt as presented above is used in the preparation of a medicament for treatment of breast cancer.

All publications and patent documents cited herein are incorporated herein by reference as if each such publication or document was specifically and individually indicated to be incorporated herein by reference. Where the text of this disclosure and the text of one or more documents incorporated by reference conflicts, this disclosure controls. Citation of publications and patent documents is not intended as an admission that any is pertinent prior art, nor does it constitute any admission as to the contents or date of the same. The embodiments described herein having now been described by way of written description, those of skill in the art will recognize that the embodiments described herein may be practiced in a variety of embodiments and that the description and examples provided herein are for purposes of illustration and not limitation of the claims.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated or unsaturated nonaromatic hydrocarbon ring having 3 to 7 carbon atoms (e.g., C₃-C₇). Examples of cycloalkyl include, but are not limited to, cyclopropyl, cyclobutyl, cyclopentyl, cyclohexyl, cycloheptyl, cyclopentenyl, cyclohexenyl, and cycloheptenyl.

The term “heterocycloalkyl” refers to a saturated or unsaturated nonaromatic 3-8 membered monocyclic groups, 7-10 membered fused bicyclic groups (or, where indicated, groups having other specified numbers of members), having one or more heteroatoms (such as O, N, or S), unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, tetrahydrothiophene, dihydropyranyl, pyranyl, morpholinyl, 1,4-diazepanyl, 1,4-oxazepanyl, and the like.

Additional examples of heterocycloalkyl groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “optionally substituted alkyl” refers to unsubstituted alkyl or alkyl having designated substituents replacing one or more hydrogen atoms on one or more carbons of the hydrocarbon backbone. Such substituents may include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

An “arylalkyl” or an “aralkyl” moiety is an alkyl substituted with an aryl (e.g., phenylmethyl(benzyl)). An “alkylaryl” moiety is an aryl substituted with an alkyl (e.g., methylphenyl).

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “optionally substituted alkenyl” refers to unsubstituted alkenyl or alkenyl having designated substituents replacing one or more hydrogen atoms on one or more hydrocarbon backbone carbon atoms. Such substituents may include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “optionally substituted alkynyl” refers to unsubstituted alkynyl or alkynyl having designated substituents replacing one or more hydrogen atoms on one or more hydrocarbon backbone carbon atoms. Such substituents may include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

“Aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR′ wherein R′ is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., bicyclic. Non-limiting example of such aryl groups include, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline).

The cycloalkyl, heterocycloalkyl, aryl, or heteroaryl ring may be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups may also be fused with alicyclic or heterocyclic rings, which are not aromatic so as to form a multicyclic system (e.g., tetralin, methylenedioxyphenyl).

When a bond to a substituent is shown to cross a bond connecting two atoms in a ring (as shown by the examples below with substituent R), then such substituent may be bonded to any atom in the ring.

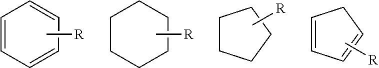

When any variable (e.g., R1) occurs more than one time in any constituent or formula for a compound, its definition at each occurrence is independent of its definition at every other occurrence. Thus, for example, if a group is shown to be substituted with 0-2 R₁ moieties, then the group may optionally be substituted with up to two R₁ moieties and R₁ at each occurrence is selected independently from the definition of R₁. The term “hydroxy” or “hydroxyl” includes groups with an —OH or —O⁻.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups may be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Calm et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Calm et al., Experientia 1956, 12, 81; Cahn, J. Chem. Educ. 1964, 41, 116).

In the present specification, each incidence of a chiral center within a structural formula, such as the non-limiting example shown here:

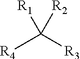

is meant to depict all possible stereoisomers. In contrast, a chiral center drawn with hatches and wedges, such as the non-limiting example shown here:

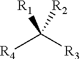

is meant to depict the stereoisomer as indicated (here in this sp³ hybridized carbon chiral center, R₃ and R₄ are in the plane of the paper, R₁ is above the plane of paper, and R₂ is behind the plane of paper).

“Geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

In the present specification, each incidence within a structural formula including a wavy line adjacent to a double bond as shown:

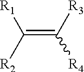

is meant to depict both geometric isomers. In contrast, such structures drawn without a wavy line is meant to depict a compound having the geometric configuration as drawn.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Where the present specification depicts a compound prone to tautomerization, but only depicts one of the tautomers, it is understood that all tautomers are included as part of the meaning of the chemical depicted. It is to be understood that the compounds disclosed herein may be depicted as different tautomers. It should also be understood that when compounds have tautomeric forms, all tautomeric forms are intended to be included, and the naming of the compounds does not exclude any tautomer form.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine.

Furthermore, the structures and other compounds disclosed herein include all atropic isomers thereof, it being understood that not all atropic isomers may have the same level of activity. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

The term “crystal polymorphs”, “polymorphs” or “crystal forms” means crystal structures in which a compound (or a salt or solvate thereof) may crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds may be prepared by crystallization under different conditions. It is understood that the compounds disclosed herein may exist in crystalline form, crystal form mixture, or anhydride or hydrate thereof.

The compounds disclosed herein include the compounds themselves, as well as their salts and solvates, if applicable. A salt, for example, may be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt may also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms.

Additionally, the compounds disclosed herein, for example, the salts of the compounds, may exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, “pharmaceutically acceptable salts” refer to derivatives of the compounds disclosed herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt may be 1:1, or any ratio other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

Chemicals as named or depicted are intended to include all naturally occurring isotopes of atoms occurring in the present compounds. Isotopes include those atoms having the same atomic number but different mass numbers. By way of general example and without limitation, isotopes of ¹H hydrogen include tritium and deuterium, and isotopes of ¹²C carbon include ¹³C and ¹⁴C.

It will be understood that some compounds, and isomers, salts, esters and solvates thereof, of the compounds disclosed herein may exhibit greater in vivo or in vitro activity than others. It will also be appreciated that some cancers may be treated more effectively than others, and may be treated more effectively in certain species of subjects that others, using the compounds, and isomers, salts, esters and solvates thereof, of the compounds disclosed herein.

As used herein, “treating” means administering to a subject a pharmaceutical composition to ameliorate, reduce or lessen the symptoms of a disease. As used herein, “treating” or “treat” describes the management and care of a subject for the purpose of combating a disease, condition, or disorder and includes the administration of a compound disclosed herein, or a pharmaceutically acceptable salt, polymorph or solvate thereof, to alleviate the symptoms or complications of a disease, condition or disorder, or to eliminate the disease, condition or disorder. The term “treat” may also include treatment of a cell in vitro or an animal model.

Treating cancer may result in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

As used herein, “subject” or “subjects” refers to any animal, such as mammals including rodents (e.g., mice or rats), dogs, primates, lemurs or humans.

Treating cancer may result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in increase in average survival time of a population of treated subjects in comparison to a population receiving monotherapy with a drug that is not a compound disclosed herein, or a pharmaceutically acceptable salt thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in a decrease in the mortality rate of a population of treated subjects in comparison to a population receiving carrier alone. Treating cancer may result in a decrease in the mortality rate of a population of treated subjects in comparison to an untreated population. Treating cancer may result in a decrease in the mortality rate of a population of treated subjects in comparison to a population receiving monotherapy with a drug that is not a compound disclosed herein, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer may result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50% even more preferably, reduced by at least 50%; and most preferably, reduced by at least 75%. Tumor growth rate may be measured by any reproducible means of measurement. Tumor growth rate may be measured according to a change in tumor diameter per unit time.

Treating cancer may result in a decrease in tumor regrowth, for example, following attempts to remove it surgically. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder may result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder may result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells may be equivalent to the mitotic index.

Treating or preventing a cell proliferative disorder may result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder may result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology may be measured by microscopy. e.g., using an inverted tissue culture microscope. An abnormal cellular morphology may take the form of nuclear pleiomorphism.

As used herein, the term “alleviate” is meant to describe a process by which the severity of a sign or symptom of a disorder is decreased. Importantly, a sign or symptom may be alleviated without being eliminated. In a preferred embodiment, the administration of pharmaceutical compositions disclosed herein leads to the elimination of a sign or symptom, however, elimination is not required. Effective dosages are expected to decrease the severity of a sign or symptom. For instance, a sign or symptom of a disorder such as cancer, which may occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the Amerimay Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of embodiments described herein, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by non-health-care professionals.

A “pharmaceutical composition” is a formulation containing a compound disclosed herein in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound disclosed herein include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The present disclosure also provides pharmaceutical compositions comprising any compound disclosed herein in combination with at least one pharmaceutically acceptable excipient or carrier.

A pharmaceutical composition disclosed herein is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH may be adjusted with acids or bases, such as hydrochloric acid or sodium hydroxide. The parenteral preparation may be enclosed in ampoules, disposable syringes or multiple dose vials made of glass or plastic.

A compound or pharmaceutical composition disclosed herein may be administered to a subject in many of the well-known methods currently used for chemotherapeutic treatment. For example, for treatment of cancers, a compound disclosed herein may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect may be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation may be determined by routine experimentation that is within the skill and judgment of the clinician. In a preferred aspect, the disease or condition to be treated is cancer. In another aspect, the disease or condition to be treated is a cell proliferative disorder.

For any compound, the therapeutically effective amount may be estimated initially either in cell culture assays, e.g., of neoplastic cells, or in animal models, usually rats, mice, rabbits, dogs, or pigs. The animal model may also be used to determine the appropriate concentration range and route of administration. Such information may then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it may be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds disclosed herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that may be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany. N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch: a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The active compounds may be prepared with pharmaceutically acceptable carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms of the compounds disclosed herein are dictated by and directly dependent on the unique characteristics of the active compound and the particular therapeutic effect to be achieved.

In therapeutic applications, the dosages of the pharmaceutical compositions used in accordance with embodiments described herein vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages may range from about 0.01 mg/kg per day to about 5000 mg/kg per day. In preferred aspects, dosages may range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day: about 0.1 mg to about 3 g/day: or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions may be included in a container, pack, or dispenser together with instructions for administration.

Techniques for formulation and administration of the compounds disclosed herein may be found in Remington: the Science and Practice of Pharmacy, 19^{th} edition, Mack Publishing Co., Easton, Pa. (1995). In an embodiment, the compounds described herein, and the pharmaceutically acceptable salts thereof, may be used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

Exemplary cancers that may be treated using one or more compounds disclosed herein include, but are not limited to, breast cancer, uterine endometrial, ovarian carcinoma, sarcoma, thyroid carcinoma, prostate, lung adenocarcinoma, and hepatocellular carcinoma.

In embodiments, the compounds disclosed herein may be useful for treating breast cancer. In embodiments, the breast cancer is ER-α+. In embodiments, the breast cancer expresses an ER-α mutation, which may be L536Q (Robinson et al. Nat Genet. 2013 December; 45(12)), L536R (Toy et al. Nat Genet. 2013 December; 45(12): 1439-45), Y537S (Toy et al. Nat Genet. 2013 December; 45(12): 1439-45; Robinson et al. Nat Genet. 2013 December; 45(12); Jeselsohn et al. Clin Cancer Res. 2014 Apr. 1; 20(7):1757-67), Y537N (Toy et al. Nat Genet. 2013 December; 45(12): 1439-45; Jeselsohn et al. Clin Cancer Res. 2014 Apr. 1; 20(7): 1757-67), Y537C (Toy et al. Nat Genet. 2013 December; 45(12):1439-45; Jeselsohn et al. Clin Cancer Res. 2014 Apr. 1; 20(7):1757-67) and D538G (Toy et al. Nat Genet. 2013 December; 45(12):1439-45; Robinson et al. Nat Genet. 2013 December; 45(12); Jeselsohn et al. Clin Cancer Res. 2014 Apr. 1; 20(7): 1757-67; Merenbakh-Lamin et al. Cancer Res. 2013 Dec. 1; 73(23):6856-64), all of which are incorporated by reference in their entireties for their teachings of ER-α mutations.

Thus, the compounds disclosed herein may be also useful for additional indications and genotypes. ESR1 mutations (Y537C/N) were recently discovered in 4 of 373 cases of endometrial cancers (Kandoth et al. Nature 2013 May 2; 497(7447):67-73; Robinson et al. Nat Genet. 2013 December; 45(12)). Since it has been shown that ESR1 mutations Y537C/N significantly drive resistance to currently marketed SOC therapies, the compounds disclosed herein may be useful for treating ERα^{MUT} endometrial cancers.

Exemplary cell proliferative disorders that may be treated using one or more compounds disclosed herein include, but are not limited to breast cancer, a precancer or precancerous condition of the breast, benign growths or lesions of the breast, and malignant growths or lesions of the breast, and metastatic lesions in tissue and organs in the body other than the breast. Cell proliferative disorders of the breast may include hyperplasia, metaplasia, and dysplasia of the breast.

A breast cancer that is to be treated may arise in a male or female subject. A breast cancer that is to be treated may arise in a premenopausal female subject or a postmenopausal female subject. A breast cancer that is to be treated may arise in a subject 30 years old or older, or a subject younger than 30 years old. A breast cancer that is to be treated has arisen in a subject 50 years old or older, or a subject younger than 50 years old. A breast cancer that is to be treated may arise in a subject 70 years old or older, or a subject younger than 70 years old.

A compound disclosed herein, or a pharmaceutically acceptable salt thereof, may be used to treat or prevent a cell proliferative disorder of the breast, or to treat or prevent breast cancer, in a subject having an increased risk of developing breast cancer relative to the population at large, or used to identify suitable candidates for such purposes. A subject with an increased risk of developing breast cancer relative to the population at large is a female subject with a family history or personal history of breast cancer. A subject with an increased risk of developing breast cancer relative to the population at large is a female who is greater than 30 years old, greater than 40 years old, greater than 50 years old, greater than 60 years old, greater than 70 years old, greater than 80 years old, or greater than 90 years old.

A cancer that is to be treated may include a tumor that has been determined to be less than or equal to about 2 centimeters in diameter. A cancer that is to be treated may include a tumor that has been determined to be from about 2 to about 5 centimeters in diameter. A cancer that is to be treated may include a tumor that has been determined to be greater than or equal to about 3 centimeters in diameter. A cancer that is to be treated may include a tumor that has been determined to be greater than 5 centimeters in diameter. A cancer that is to be treated may be classified by microscopic appearance as well differentiated, moderately differentiated, poorly differentiated, or undifferentiated. A cancer that is to be treated may be classified by microscopic appearance with respect to mitosis count (e.g., amount of cell division) or nuclear pleiomorphism (e.g., change in cells). A cancer that is to be treated may be classified by microscopic appearance as being associated with areas of necrosis (e.g., areas of dying or degenerating cells). A cancer that is to be treated may be classified as having an abnormal karyotype, having an abnormal number of chromosomes, or having one or more chromosomes that are abnormal in appearance. A cancer that is to be treated may be classified as being aneuploid, triploid, tetraploid, or as having an altered ploidy. A cancer that is to be treated may be classified as having a chromosomal translocation, or a deletion or duplication of an entire chromosome, or a region of deletion, duplication or amplification of a portion of a chromosome.

The compounds, or pharmaceutically acceptable salts thereof may be administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds may be selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian may readily determine and prescribe the effective amount of the drug required to prevent, counter, or arrest the progress of the condition.

### EXAMPLES

Hereby are provided non-limiting examples of embodiments of compounds disclosed herein. If there is any discrepancy between a compound's depicted chemical structure and its chemical name, the depicted chemical structure will control.

TABLE 1
Compounds

| Compound | | |
|---|---|---|
| # | Structure | Name |
| 101 | 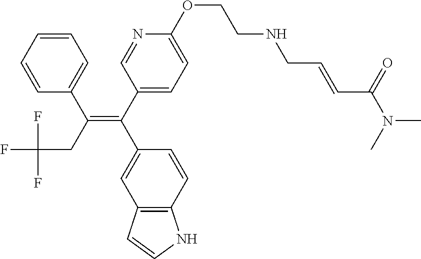 [SMILES: CN(C)C(=O)/C=C/CNCCOc1ccc(/C(=C(/CC(F)(F)F)c2ccccc2)c2ccc3[nH]ccc3c2)cn1] | (2E)-N,N- dimethyl-4-[[2- ([5-[(1E)-4,4,4- trifluoro-1-(1H- indol-5-yl)-1- phenylbut-1-en- 2-yl]pyridin-2- yl]oxy)ethyl] amino]but-2- enamide |
| 102 | 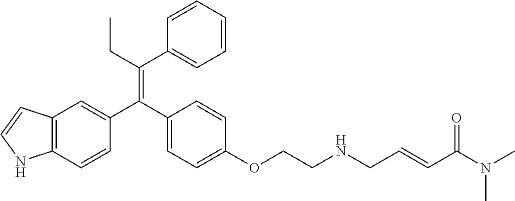 [SMILES: CC/C(=C(/c1ccc(OCCNC/C=C/C(=O)N(C)C)cc1)c1ccc2[nH]ccc2c1)c1ccccc1] | (E)-4-(2- (4-((E)-1-(1H- indol-5-yl)-2- phenylbut-1- enyl)phenoxy) ethyl amino)-N,N- dimethylbut-2- enamide |
| 103 | 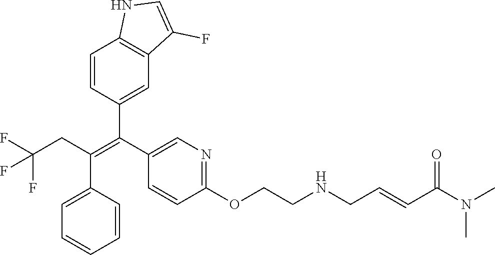 [SMILES: CN(C)C(=O)/C=C/CNCCOc1ccc(/C(=C(/CC(F)(F)F)c2ccccc2)c2ccc3[nH]cc(F)c3c2)cn1] | (E)-N,N- dimethyl-4-((2- ((5-((Z)-4,4,4- trifluoro-1-(3- fluoro-1H-indol- 5-yl)-2- phenylbut-1-en- 1-yl)pyridin-2- yl)oxy)ethyl) amino)but-2- enamide |
| 104 | 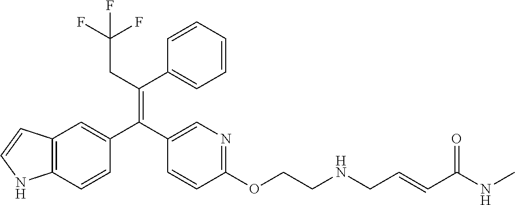 [SMILES: CNC(=O)/C=C/CNCCOc1ccc(/C(=C(/CC(F)(F)F)c2ccccc2)c2ccc3[nH]ccc3c2)cn1] | (E)-N-methyl-4- ((2-((5-((Z)- 4,4,4-trifluoro-1- (1H-indol-5-yl)- 2-phenylbut-1- en-1-yl)pyridin- 2- yl)oxy)ethyl) amino)but-2- enamide |
| 105 | 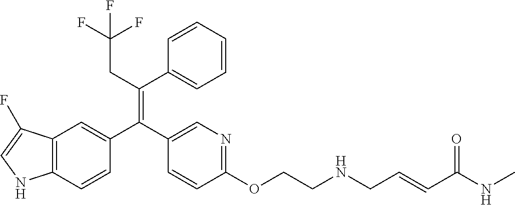 [SMILES: CNC(=O)/C=C/CNCCOc1ccc(/C(=C(/CC(F)(F)F)c2ccccc2)c2ccc3[nH]cc(F)c3c2)cn1] | (E)-N-methyl-4- ((2-((5-((Z)- 4,4,4-trifluoro-1- (3-fluoro-1H- indol-5-yl)-2- phenylbut-1-en- 1-yl)pyridin-2- yl)oxy)ethyl) amino)but-2- enamide |
| 106 | 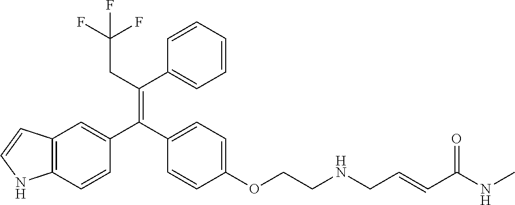 [SMILES: CNC(=O)/C=C/CNCCOc1ccc(/C(=C(/CC(F)(F)F)c2ccccc2)c2ccc3[nH]ccc3c2)cc1] | (E)-N-methyl-4- ((2-(4-((E)-4,4,4- trifluoro-1-(1H- indol-5-yl)-2- phenylbut-1-en- 1- yl)phenoxy)ethyl) amino)but-2- enamide |
| 107 | 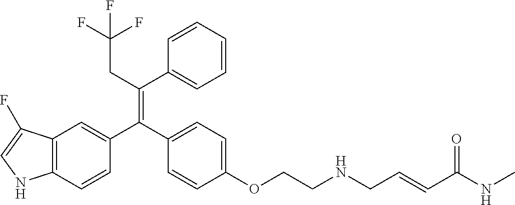 [SMILES: CNC(=O)/C=C/CNCCOc1ccc(/C(=C(/CC(F)(F)F)c2ccccc2)c2ccc3[nH]cc(F)c3c2)cc1] | (E)-N-methyl-4- ((2-(4-((E)-4,4,4- trifluoro-1-(3- fluoro-1H-indol- 5-yl)-2- phenylbut-1-en- 1- yl)phenoxy)ethyl) amino)but-2- enamide |
| 108 | 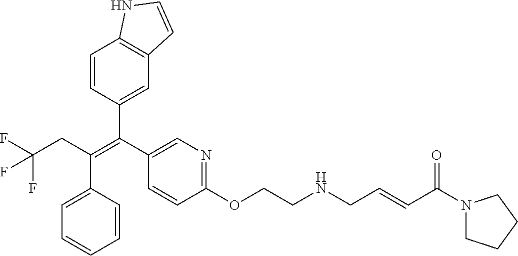 [SMILES: O=C(/C=C/CNCCOc1ccc(/C(=C(/CC(F)(F)F)c2ccccc2)c2ccc3[nH]ccc3c2)cn1)N1CCCC1] | (E)-1-(pyrrolidin- 1-yl)-4-((2-((5- ((Z)-4,4,4- trifluoro-1-(1H- indol-5-yl)-2- phenylbut-1-en- 1-yl)pyridin-2- yl)oxy)ethyl) amino)but-2-en- 1-one |
| 109 | 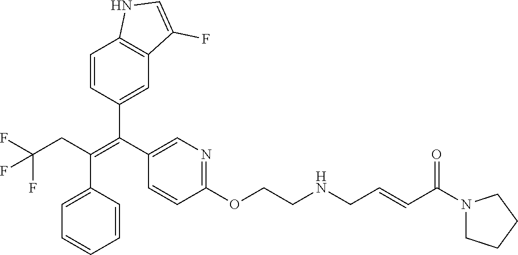 [SMILES: O=C(/C=C/CNCCOc1ccc(/C(=C(/CC(F)(F)F)c2ccccc2)c2ccc3[nH]cc(F)c3c2)cn1)N1CCCC1] | (E)-1-(pyrrolidin- 1-yl)-4-((2-((5- ((Z)-4,4,4- trifluoro-1-(3- fluoro-1H-indol- 5-yl)-2- phenylbut-1-en- 1-yl)pyridin-2- yl)oxy)ethyl) amino)but-2-en- 1-one |
| 110 | 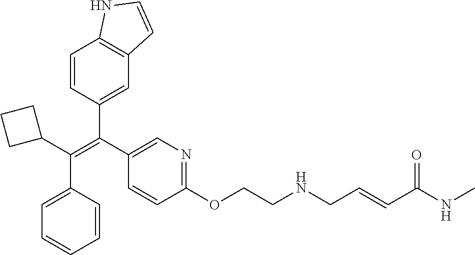 [SMILES: CNC(=O)/C=C/CNCCOc1ccc(/C(=C(\c2ccccc2)C2CCC2)c2ccc3[nH]ccc3c2)cn1] | (E)-4-((2-((5- ((Z)-2- cyclobutyl-1- (1H-indol-5-yl)- 2- phenylvinyl) pyridin-2- yl)oxy)ethyl) amino)-N- methylbut- 2-enamide |
| 111 | 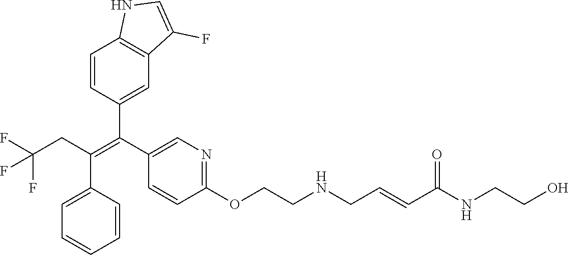 [SMILES: O=C(/C=C/CNCCOc1ccc(/C(=C(/CC(F)(F)F)c2ccccc2)c2ccc3[nH]cc(F)c3c2)cn1)NCCO] | (E)-N-(2- hydroxyethyl)-4- ((2-((5-((Z)- 4,4,4-trifluoro-1- (3-fluoro-1H- indol-5-yl)-2- phenylbut-1-en- 1-yl)pyridin-2- yl)oxy)ethyl) amino)but-2- enamide |
| 112 | 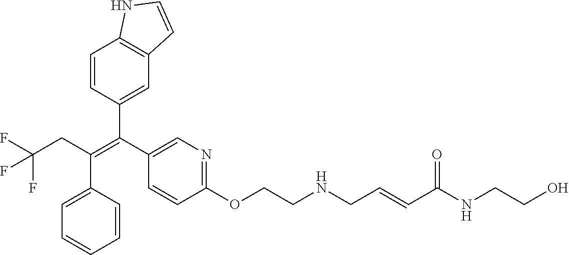 [SMILES: O=C(/C=C/CNCCOc1ccc(/C(=C(/CC(F)(F)F)c2ccccc2)c2ccc3[nH]ccc3c2)cn1)NCCO] | (E)-N-(2- hydroxyethyl)-4- ((2-((5-((Z)- 4,4,4-trifluoro-1- (1H-indol-5-yl)- 2-phenylbut-1- en-1-yl)pyridin- 2- yl)oxy)ethyl) amino)but-2- enamide |
| 113 | 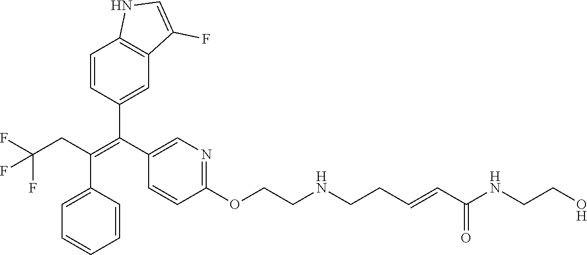 [SMILES: O=C(/C=C/CCNCCOc1ccc(/C(=C(/CC(F)(F)F)c2ccccc2)c2ccc3[nH]cc(F)c3c2)cn1)NCCO] | (E)-N-(2- hydroxyethyl)-5- ((2-((5-((Z)- 4,4,4-trifluoro-1- (3-fluoro-1H- indol-5-yl)-2- phenylbut-1-en- 1-yl)pyridin-2- yl)oxy)ethyl) amino)pent-2- enamide |
| 114 | 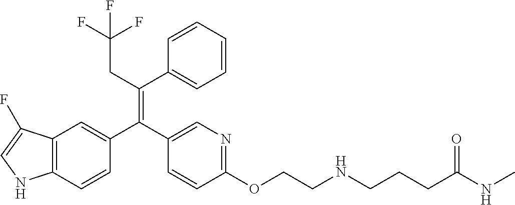 [SMILES: CNC(=O)CCCNCCOc1ccc(/C(=C(/CC(F)(F)F)c2ccccc2)c2ccc3[nH]cc(F)c3c2)cn1] | (Z)-N-methyl-4- ((2-((5-(4,4,4- trifluoro-1-(3- fluoro-1H-indol- 5-yl)-2- phenylbut-1-en- 1-yl)pyridin-2- yl)oxy)ethyl) amino)butanamide |
| 115 | 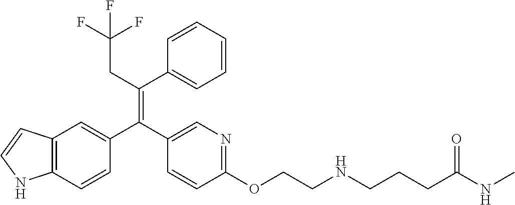 [SMILES: CNC(=O)CCCNCCOc1ccc(/C(=C(/CC(F)(F)F)c2ccccc2)c2ccc3[nH]ccc3c2)cn1] | (Z)-N-methyl-4- ((2-((5-(4,4,4- trifluoro-1-(1H- indol-5-yl)-2- phenylbut-1-en- 1-yl)pyridin-2- yl)oxy)ethyl) amino) butanamide |
| 116 | 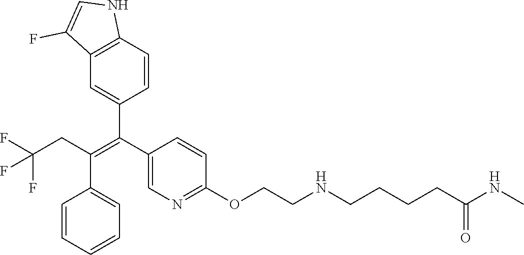 [SMILES: CNC(=O)CCCCNCCOc1ccc(/C(=C(/CC(F)(F)F)c2ccccc2)c2ccc3[nH]cc(F)c3c2)cn1] | (Z)-N-methyl-5- ((2-((5-(4,4,4- trifluoro-1-(3- fluoro-1H-indol- 5-yl)-2- phenylbut-1-en- 1-yl)pyridin-2- yl)oxy)ethyl) amino) pentanamide |
| 117 | 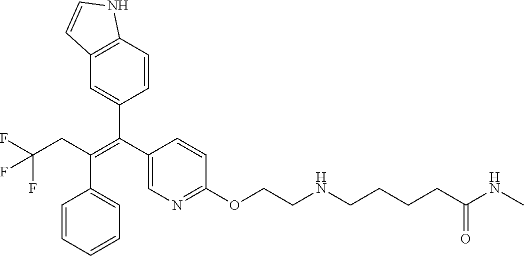 [SMILES: CNC(=O)CCCCNCCOc1ccc(/C(=C(/CC(F)(F)F)c2ccccc2)c2ccc3[nH]ccc3c2)cn1] | (Z)-N-methyl-5- ((2-((5-(4,4,4- trifluoro-1-(1H- indol-5-yl)-2- phenylbut-1-en- 1-yl)pyridin-2- yl)oxy)ethyl) amino) pentanamide |
| 118 | 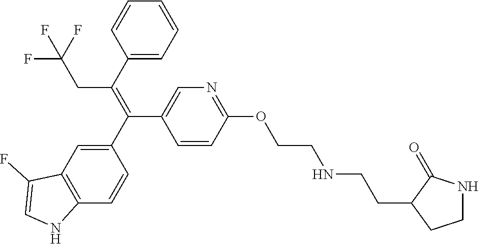 [SMILES: O=C1NCCC1CCNCCOc1ccc(/C(=C(/CC(F)(F)F)c2ccccc2)c2ccc3[nH]cc(F)c3c2)cn1] | (Z)-3-(2-((2-((5- (4,4,4-trifluoro- 1-(3-fluoro-1H- indol-5-yl)-2- phenylbut-1-en- 1-yl)pyridin-2- yl)oxy)ethyl) amino)ethyl) pyrrolidin-2-one |
| 119 | 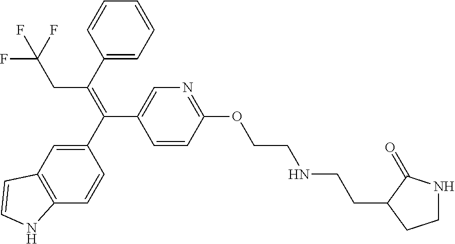 [SMILES: O=C1NCCC1CCNCCOc1ccc(/C(=C(/CC(F)(F)F)c2ccccc2)c2ccc3[nH]ccc3c2)cn1] | (Z)-3-(2-((2-((5- (4,4,4-trifluoro- 1-(1H-indol-5- yl)-2-phenylbut- 1-en-1- yl)pyridin-2- yl)oxy)ethyl) amino)ethyl) pyrrolidin-2-one |
| 120 | 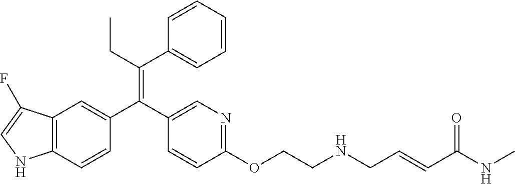 [SMILES: CC/C(=C(/c1ccc(OCCNC/C=C/C(=O)NC)nc1)c1ccc2[nH]cc(F)c2c1)c1ccccc1] | (E)-4-((2-((5- ((Z)-1-(3-fluoro- 1H-indol-5-yl)-2- phenylbut-1-en- 1-yl)pyridin-2- yl)oxy)ethyl) amino)-N- methylbut- 2-enamide |
| 121 | 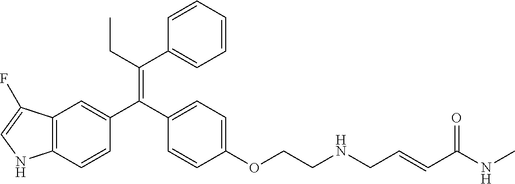 [SMILES: CC/C(=C(/c1ccc(OCCNC/C=C/C(=O)NC)cc1)c1ccc2[nH]cc(F)c2c1)c1ccccc1] | (E)-4-((2-(4-((E)- 1-(3-fluoro-1H- indol-5-yl)-2- phenylbut-1-en- 1- yl)phenoxy)ethyl) amino)-N- methylbut-2- enamide |
| 122 | 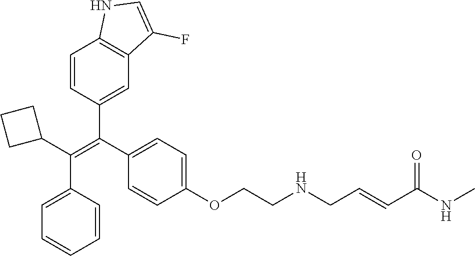 [SMILES: CNC(=O)/C=C/CNCCOc1ccc(/C(=C(\c2ccccc2)C2CCC2)c2ccc3[nH]cc(F)c3c2)cc1] | (E)-4-((2-(4-((E)- 2-cyclobutyl-1- (3-fluoro-1H- indol-5-yl)-2- phenylvinyl) phenoxy)ethyl) amino)-N- methylbut-2- enamide |
| 123 | 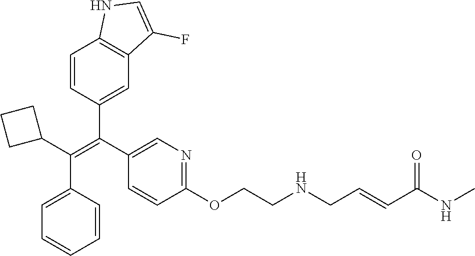 [SMILES: CNC(=O)/C=C/CNCCOc1ccc(/C(=C(\c2ccccc2)C2CCC2)c2ccc3[nH]cc(F)c3c2)cn1] | (E)-4-((2-((5- ((Z)-2- cyclobutyl-1-(3- fluoro-1H-indol- 5-yl)-2- phenylvinyl) pyridin-2- yl)oxy)ethyl) amino)-N- methylbut- 2-enamide |
| 124 | 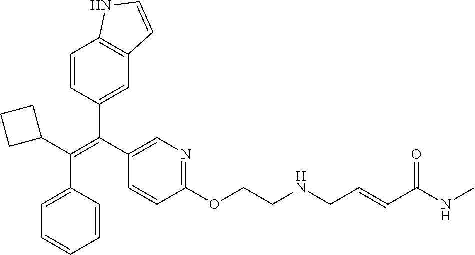 [SMILES: CNC(=O)/C=C/CNCCOc1ccc(/C(=C(\c2ccccc2)C2CCC2)c2ccc3[nH]ccc3c2)cn1] | (E)-4-((2-((5- ((Z)-2- cyclobutyl-1- (1H-indol-5-yl)- 2- phenylvinyl) pyridin-2- yl)oxy)ethyl) amino)-N- methylbut- 2-enamide |

TABLE 2
Viability Screen Results (see Example 201 below)

| | WT ER | | | | Y537S | Y537S |
|---|---|---|---|---|---|---|
| | CTGlo | | WT ER | Y537S ER | ER | ER |
| | MCF7.6 | WT ER | CTGlo | CTGlo | CTGlo | CTGlo |
| | GI50 | CTGlo | MCF7.6 | MCF7.7 | MCF7.7 | MCF7.7 |
| Compound | Mean | MCF7.6 | GI50 | GI50 Mean | GI50 | GI50 |
| # | (nM) | GI50 SD | Count | (nM) | SD | Count |
| 1 | 0.71 | 0.22 | 2 | 3.05 | 2.38 | 2 |
| 2 | 1.75 | 1.21 | 7 | 27.47 | 19.29 | 7 |

### General Procedures

The following abbreviations may be used herein:
ACN: Acetonitrile
BOC: tert-butyloxycarbonyl
CAN: ceric ammonium nitrate
Conc.: concentrated
Cs₂CO₃: Cesium carbonate
DABCO: 1,4-Diazabicyclo[2.2.2]octane
DCM: Dichloromethane
DHP: Dihydropyran
DIPEA: N,N-diisopropylethylamine, Hunig's base
DMA: Dimethylacetamide
DMF: Dimethylformamide
DMSO: dimethylsulfoxide
DPEphos: (Oxydi-2,1-phenylene)bis(diphenylphosphine)
EDCl.HCl: N-(3-Dimethylaminopropyl)-N-ethylcarbodiimide hydrochloride
EtOH: Ethanol
EtOAc: Ethyl acetate
Et₃N: Triethylamine
Ex.: Example
h: Hours
HATU: 1-[Bis(dimethylamino)methylene]-1H-1,2,3-triazolo[4,5-b]pyridinium 3-oxid hexafluorophosphate
HCl: Hydrochloric acid
HMPA: Hexamethylphosphoramide
HPLC: High-performance liquid chromatography
H₂SO₄: Sulfuric acid
IPA: Isopropyl alcohol
K2CO3: Potassium carbonate
KOH: Potassium hydroxide
LCMS: Liquid chromatography-mass spectrometry
MeOH: Methanol
Na₂CO₃: Sodium carbonate
NBS: n-bromosuccinimide
nBuLi: n-Butyllithium
NH₄Cl: Ammonium chloride
NH₄OH: Ammonium hydroxide
NMR: nuclear magnetic resonance
on or o.n.: overnight
Pd/C: Palladium (0) on carbon
Pd₂(dba)₃: Tris(dibenzylideneacetone)dipalladium(0)
PPTS: pyridinium p-toluenesulfonate
PTSA: p-toluenesulfonic acid
RT or r.t.: room temperature
TBAF: Tetrabutylammonium fluoride
TEA: Triethylamine
TFA: Trifluoroacetic acid
THF: Tetrahydrofuran
TLC: Thin-layer chromatography
Pt/C: Platinum (0) on carbon

Unless indicated otherwise, ¹H NMR spectra were taken on a Bruker 300 MHz or 400 MHz NMR.

### Examples

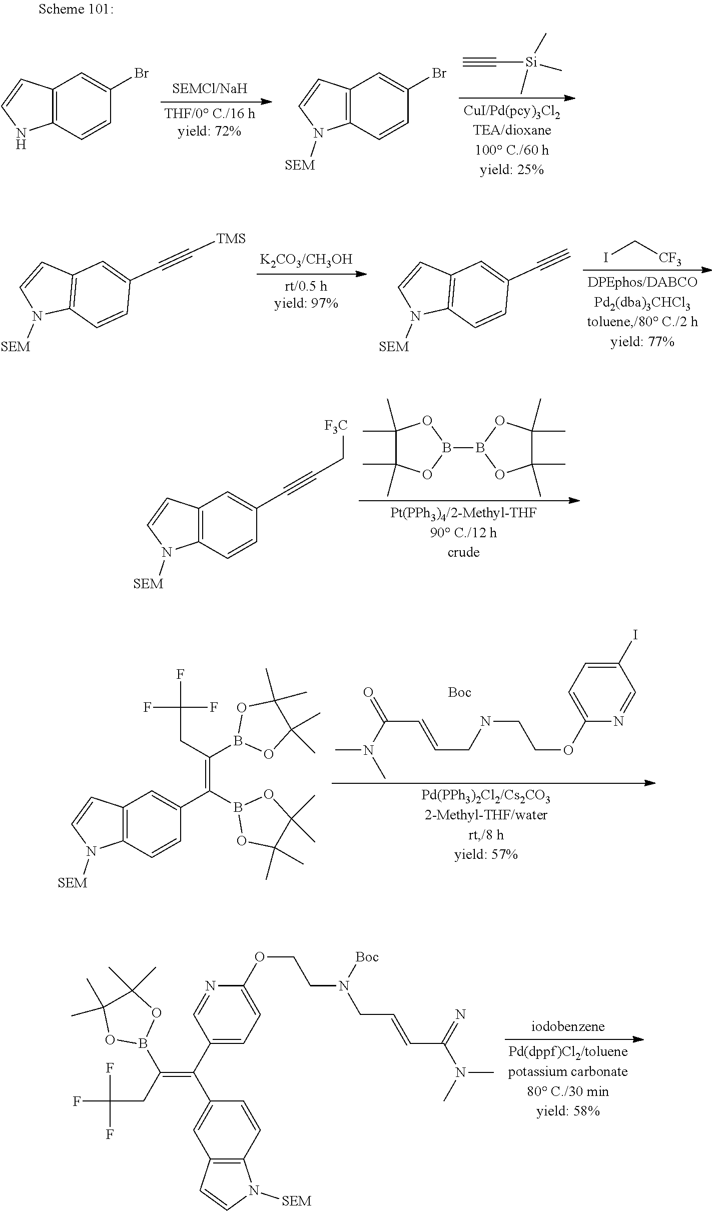

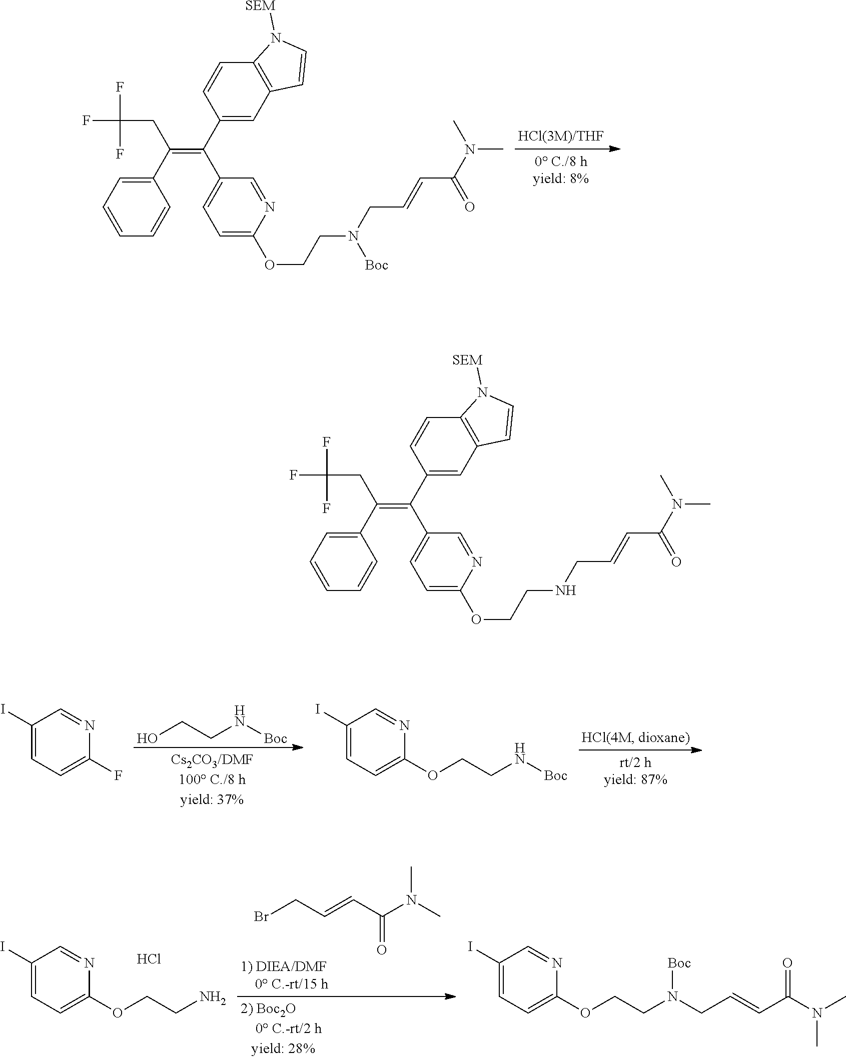

### Example 101: Synthesis of (2E)-N,N-dimethyl-4-[[2-([5-[(1E)-4,4,4-trifluoro-1-(1H-indol-5-yl)-1-phenylbut-1-en-2-yl]pyridin-2-yl]oxy)ethyl]amino]but-2-enamide (Compound 101)

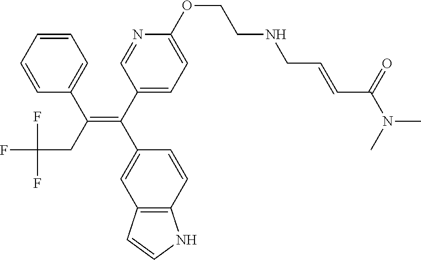 [SMILES: CN(C)C(=O)/C=C/CNCCOc1ccc(/C(=C(/CC(F)(F)F)c2ccccc2)c2ccc3[nH]ccc3c2)cn1]

### Step-1: Synthesis of 5-bromo-1-[[2-(trimethylsilyl)ethoxy]methyl]-1H-indole

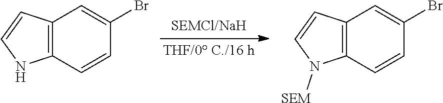 [SMILES: Brc1ccc2[nH]ccc2c1 ; C[Si](C)(C)CCOCn1ccc2cc(Br)ccc21]

Into a 500-mL 3-necked round-bottom flask purged and maintained with an inert atmosphere of nitrogen, was placed 5-bromo-1H-indole (15 g, 76.51 mmol, 1.00 equiv), THF (200 mL). This was followed by the addition of sodium hydride (4 g, 166.67 mmol, 1.30 equiv) in portions at 0° C. To this was added SEMCl (14.05 g, 92.01 mmol, 1.10 equiv) dropwise with stirring at 0° C. The resulting solution was stirred for 16 h at 25 degrees C. The reaction mixture was cooled with a water/ice bath and quenched by the addition of 50 mL of NH₄Cl (sat. aq.). The resulting solution was extracted with 3×200 mL of ethyl acetate and the organic layers combined and dried over anhydrous sodium sulfate and concentrated under vacuum. The residue was applied onto a silica gel column with ethyl acetate/petroleum ether (2:98). This resulted in 18 g (72%) of 5-bromo-1-[[2-(trimethylsilyl)ethoxy]methyl]-1H-indole as off-white oil.

### Step-2: Synthesis of 1-[[2-(trimethylsilyl)ethoxy]methyl]-5-[2-(trimethylsilyl)ethynyl]-1H-indole

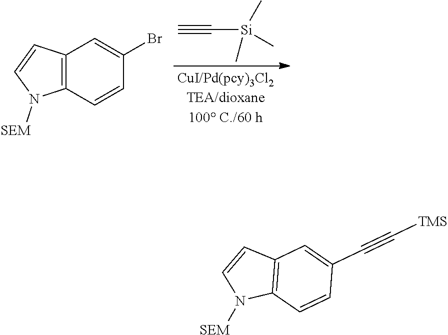

Into a 250-mL pressure tank reactor purged and maintained with an inert atmosphere of nitrogen, was placed ethynyltrimethylsilane (31.5 g, 320.71 mmol, 5.00 equiv), 5-bromo-1-[[2-(trimethylsilyl)ethoxy]methyl]-1H-indole (21 g, 64.36 mmol, 1.00 equiv), TEA (12.9 g, 127.48 mmol, 2.00 equiv), CuI (3.57 g, 18.75 mmol, 0.30 equiv), Pd(PCy)₃Cl₂ (7.09 g, 9.63 mmol, 0.15 equiv), dioxane (60 mL). The resulting solution was stirred for 60 h at 100 degrees C. in an oil bath. The resulting solution was extracted with 3×300 mL of ethyl acetate, washed with 300 mL brine. Then the organic layers was combined and dried over anhydrous sodium sulfate and concentrated under vacuum. The residue was applied onto a silica gel column with EA:PE (10:90). This resulted in 5.5 g (25%) of 1-[[2-(trimethylsilyl)ethoxy]methyl]-5-[2-(trimethylsilyl)ethynyl]-1H-indole as yellow oil.

### Step-3: Synthesis of S-ethynyl-1-[[2-(trimethylsilyl)ethoxy]methyl]-1H-indole

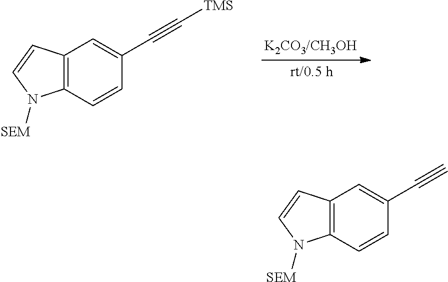 [SMILES: C[Si](C)(C)C#Cc1ccc2c(ccn2COCC[Si](C)(C)C)c1 ; C#Cc1ccc2c(ccn2COCC[Si](C)(C)C)c1]

Into a 100-mL round-bottom flask, was placed 1-[[2-(trimethylsilyl)ethoxy]methyl]-5-[2-(trimethylsilyl)ethynyl]-1H-indole (5.5 g, 16.01 mmol, 1.00 equiv), methanol (60 mL), potassium carbonate (4.4 g, 31.84 mmol, 2.00 equiv). The resulting solution was stirred for 0.5 h at room temperature. The resulting solution was extracted with 3×100 mL of ethyl acetate and the organic layers combined and dried over anhydrous sodium sulfate and concentrated under vacuum. The residue was applied onto a silica gel column with ethyl acetate/petroleum ether (2:98). This resulted in 4.2 g (97%) of 5-ethynyl-1-[[2-(trimethylsilyl)ethoxy]methyl]-1H-indole as yellow oil.

### Step-4: Synthesis of 5-(4,4,4-trifluorobut-1-yn-1-yl)-1-[[2-(trimethylsilyl)ethoxy]methyl]-1H-indole

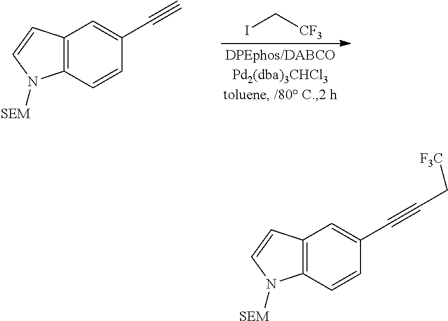 [SMILES: C#Cc1ccc2c(ccn2COCC[Si](C)(C)C)c1 ; FC(F)(F)CI ; C[Si](C)(C)CCOCn1ccc2cc(C#CCC(F)(F)F)ccc21]

Into a 40-mL vial purged and maintained with an inert atmosphere of nitrogen, was placed 5-ethynyl-1-[[2-(trimethylsilyl)ethoxy]methyl]-1H-indole (2.0 g, 7.37 mmol, 1.00 equiv), 1,1,1-trifluoro-2-iodoethane (8.2 g, 39.06 mmol, 5.00 equiv), toluene (20 mL), DPEphos (840 mg, 0.20 equiv), Pd₂(dba)₃CHCl₃ (400 mg, 0.05 equiv), DABCO (1.72 g, 2.00 equiv). The resulting solution was stirred for 2 h at 80 degrees C. in an oil bath. The resulting mixture was quenched with water (100 mL) and extracted with EA (100 mL×3) then washed with brine (100 mL) and concentrated under vacuum. The residue was applied onto a silica gel column with ethyl acetate/petroleum ether (5:95). This resulted in 2.0 g (77%) of 5-(4,4,4-trifluorobut-1-yn-1-yl)-1-[[2-(trimethylsilyl)ethoxy]methyl]-1H-indole as yellow oil.

### Step-5: Synthesis of 5-[4,4,4-trifluoro-1,2-bis(tetramethyl-1,3,2-dioxaborolan-2-yl)but-1-en-1-yl]-1-[[2-(trimethylsilyl)ethoxy]methyl]-1H-indole

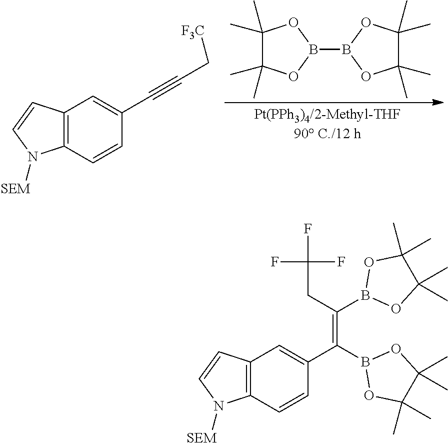

Into a 40-mL round-bottom flask purged and maintained with an inert atmosphere of nitrogen, was placed 5-(4,4,4-trifluorobut-1-yn-1-yl)-1-[[2-(trimethylsilyl)ethoxy]methyl]-1H-indole (353 mg, 1.00 mmol, 1.00 equiv), Pt(PPh3)4 (12.4 mg, 0.01 equiv), 2-Me-THF (5 mL), 4,4,5,5-tetramethyl-2-(tetramethyl-1,3,2-dioxaborolan-2-yl)-1,3,2-dioxaborolane (254 mg, 1.00 mmol, 1.00 equiv). The resulting solution was stirred for 12 h at 90 degrees C. in an oil bath. The resulting mixture was concentrated under vacuum. This resulted in 608 mg (crude) of 5-[4,4,4-trifluoro-1,2-bis(tetramethyl-1,3,2-dioxaborolan-2-yl)but-1-en-1-yl]-1-[[2-(trimethylsilyl)ethoxy]methyl]-1H-indole as a brown solid.

### Step-6: Synthesis of tert-butyl N-[2-[(5-iodopyridin-2-yl)oxy]ethyl]carbamate

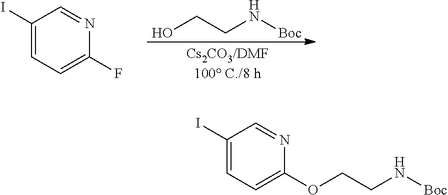 [SMILES: Fc1ccc(I)cn1 ; CC(C)(C)OC(=O)NCCO ; CC(C)(C)OC(=O)NCCOc1ccc(I)cn1]

Into a 100-mL round-bottom flask, was placed 2-fluoro-5-iodopyridine (5 g, 22.42 mmol, 1.00 equiv), tert-butyl N-(2-hydroxyethyl)carbamate (5.4 g, 33.50 mmol, 1.50 equiv), Cs₂CO₃ (13 g, 3.00 equiv), N,N-dimethylformamide (30 mL). The resulting solution was stirred for 8 h at 100° C. in an oil bath. Then the reaction mixture was poured into ice cold water, the solid was filtered and dried under reduced pressure to obtain 3.0 g (37%) of tert-butyl N-[2-[(5-iodopyridin-2-yl)oxy]ethyl]carbamate as a white solid.

### Step-7: Synthesis of 2-(2-aminoethoxy)-5-iodopyridine Hydrochloride

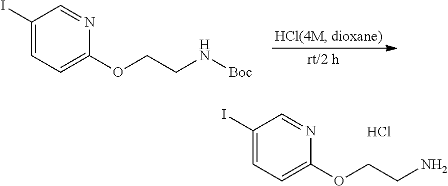

Into a 100-mL round-bottom flask, was placed tert-butyl N-[2-[(5-iodopyridin-2-yl)oxy]ethyl]carbamate (5.6 g, 15.38 mmol, 1.00 equiv), hydrogen chloride saturated dioxane solution (4M, 20 mL). The resulting solution was stirred for 2 h at room temperature. The resulting mixture was concentrated under vacuum. This resulted in 4.0 g (87%) of 2-(2-aminoethoxy)-5-iodopyridine hydrochloride as a white solid.

### Step-8: Synthesis of tert-butyl N-[(2E)-3-(dimethylcarbamoyl)prop-2-en-1-yl]-N-[2-[(5-iodopyridin-2-yl)oxy]ethyl]carbamate

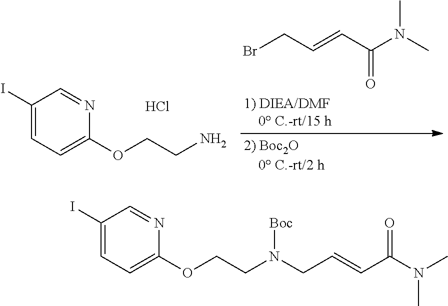

Into a 100-mL round-bottom flask, was placed 2-(2-aminoethoxy)-5-iodopyridine (2 g, 7.57 mmol, 1.00 equiv), DIEA (1.95 g, 2.00 equiv), N,N-dimethylformamide (10 mL). This was followed by the addition of (2E)-4-bromo-N,N-dimethylbut-2-enamide (1.02 g, 5.31 mmol, 0.70 equiv) dropwise with stirring at 0° C. The resulting solution was stirred for 30 min at 0° C. in an ice/salt bath. The resulting solution was allowed to react, with stirring, for an additional 5 h at room temperature. To this was added (Boc)₂O (1.8 g, 1.20 equiv). The resulting solution was stirred for 2 h at 0° C. in an ice/salt bath. The reaction mixture was diluted with ice cold water (100 mL) and extracted with EA (3*100 mL). The organic layer was combined, washed with brine (100 mL), dried over anhydrous sodium sulfate and concentrated under reduced pressure. The crude material was purified by column chromatography with silica gel column using 80% ethyl acetate in n-hexane as an eluent to afford 1.0 g (28%) of tert-butyl N-[(2E)-3-(dimethylcarbamoyl)prop-2-en-1-yl]-N-[2-[(5-iodopyridin-2-yl)oxy]ethyl]carbamate as colorless oil.

### Step-9: Synthesis of Tert-butyl N-[(2E)-3-(dimethylcarbamoyl)prop-2-en-1-yl]-N-[2-([5-[(1E)-4,4,4-trifluoro-2-(tetramethyl-1,3,2-dioxaborolan-2-yl)-1-(1-[[2-(trimethylsilyl)ethoxy]methyl]-1H-indol-5-yl)but-1-en-1-yl]pyridin-2-yl]oxy)ethyl]carbamate

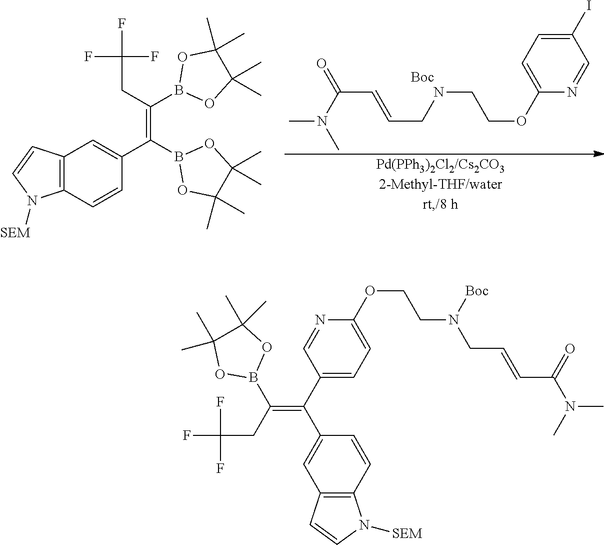

Into a 40-mL round-bottom flask purged and maintained with an inert atmosphere of nitrogen, was placed 5-[(1Z)-4,4,4-trifluoro-1,2-bis(tetramethyl-1,3,2-dioxaborolan-2-yl)but-1-en-1-yl]-1-[[2-(trimethylsilyl)ethoxy]methyl]-1H-pyrrolo[2,3-c]pyridine (911 mg, 1.50 mmol, 1.00 equiv), tert-butyl N-[(2E)-3-(dimethylcarbamoyl)prop-2-en-1-yl]-N-[2-[(5-iodopyridin-2-yl)oxy]ethyl]carbamate (713 mg, 1.50 mmol, 1.00 equiv), Pd(PPh₃)₂Cl₂ (53 mg, 0.08 mmol, 0.05 equiv), Cs₂CO₃ (1.23 g, 3.78 mmol, 2.50 equiv), 2-Methyl-THF (10 mL), water (2 mL). The resulting solution was stirred for 8 h at room temperature. The resulting mixture was quenched with water/ice (100 mL) and extracted with EA (100 mL*3) then washed with brine (100 mL) and concentrated under vacuum. The crude product was purified by C18 column, mobile phase, H₂O/CH₃CN=3/7; Detector, UV 254 nm. This resulted in 710 mg (57%) of tert-butyl N-[(2E)-3-(dimethylcarbamoyl)prop-2-en-1-yl]-N-[2-([5-[(1E)-4,4,4-trifluoro-2-(tetramethyl-1,3,2-dioxaborolan-2-yl)-1-(1-[[2-(trimethylsilyl)ethoxy]methyl]-1H-indol-5-yl)but-1-en-1-yl]pyridin-2-yl]oxy)ethyl]carbamate as a off-white solid.

### Step-10: Synthesis of Tert-butyl N-[(2E)-3-(dimethylcarbamoyl)prop-2-en-1-yl]-N-[2-([5-[(1Z)-4,4,4-trifluoro-2-phenyl-1-(1-[[2-(trimethylsilyl)ethoxy]methyl]-1H-indol-5-yl)but-1-en-1-yl]pyridin-2-yl]oxy)ethyl]carbamate

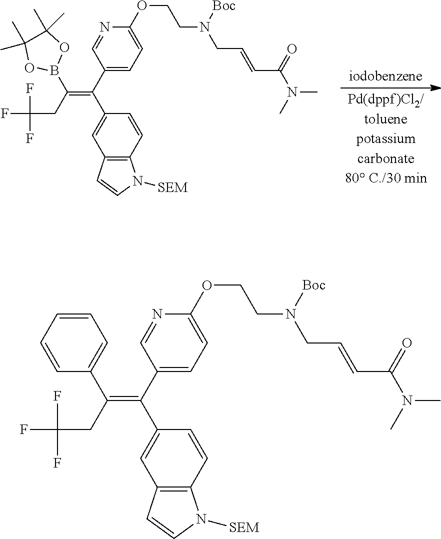

Into a 40-mL round-bottom flask purged and maintained with an inert atmosphere of nitrogen, was placed tert-butyl N-[(2E)-3-(dimethylcarbamoyl)prop-2-en-1-yl]-N-[2-([5-[(1E)-4,4,4-trifluoro-2-(tetramethyl-1,3,2-dioxaborolan-2-yl)-1-(1-[[2-(trimethylsilyl)ethoxy]methyl]-1H-indol-5-yl)but-1-en-1-yl]pyridin-2-yl]oxy)ethyl]carbamate (366 mg, 0.44 mmol, 1.00 equiv), iodobenzene (270 mg, 1.32 mmol, 3.00 equiv), Pd(dppf)Cl₂ (32 mg, 0.04 mmol, 0.10 equiv), potassium carbonate (304 mg, 2.20 mmol, 5.00 equiv), toluene (10 mL), water (2 mL). The resulting solution was stirred for 30 min at 80 degrees C. in an oil bath. The reaction was then quenched by the addition of 100 mL of water. The resulting solution was extracted with 3×100 mL of ethyl acetate and the organic layers combined and concentrated under vacuum. The crude product was purified by C18 Column, mobile phase, water/CH₃CN=3/2. This resulted in 200 mg (58%) of tert-butyl N-[(2E)-3-(dimethylcarbamoyl)prop-2-en-1-yl]-N-[2-([5-[(1 Z)-4,4,4-trifluoro-2-phenyl-1-(1-[[2-(trimethylsilyl)ethoxy]methyl]-1H-indol-5-yl)but-1-en-1-yl]pyridin-2-yl]oxy)ethyl]carbamate as an off-white solid.

### Step-11: Synthesis of (2E)-N,N-dimethyl-4-[[2-([5-[(1E)-4,4,4-trifluoro-1-(1H-indol-5-yl)-1-phenylbut-1-en-2-yl]pyridin-2-yl]oxy)ethyl]amino]but-2-enamide

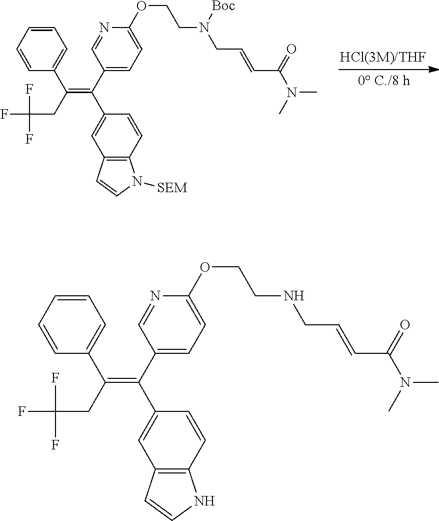 [SMILES: CN(C)C(=O)/C=C/CN(CCOc1ccc(/C(=C(/CC(F)(F)F)c2ccccc2)c2ccc3c(ccn3COCC[Si](C)(C)C)c2)cn1)C(=O)OC(C)(C)C ; CN(C)C(=O)/C=C/CNCCOc1ccc(/C(=C(/CC(F)(F)F)c2ccccc2)c2ccc3[nH]ccc3c2)cn1]

Into a 100-mL round-bottom flask, was placed tert-butyl N-[(2E)-3-(dimethylcarbamoyl)prop-2-en-1-yl]-N-[2-([5-[(1E)-4,4,4-trifluoro-1-phenyl-1-(1-[[2-(trimethylsilyl)ethoxy]methyl]-1H-indol-5-yl)but-1-en-2-yl]pyridin-2-yl]oxy)ethyl]carbamate (200 mg, 0.26 mmol, 1.00 equiv), tetrahydrofuran (20 mL), hydrochloric acid (3M, 20 mL). The resulting solution was stirred for 8 h at 0 degrees C. in a water/ice bath. The pH value of the solution was at first adjusted to 8 with Na₂CO₃(sat 20 mL), then adjusted to 12 with sodium hydroxide (sat. 20 mL). The resulting solution was extracted with 3×20 mL of ethyl acetate and the organic layers combined and concentrated under vacuum. The crude product was purified by Prep-HPLC with the following conditions (1#-Waters 2767-1): Column, X-bridge Prep phenyl 5 um, 19*150 mmh; mobile phase, Phase A: water with 0.5% NH₄HCO₃ Phase B: CH₃CN. Water with 0.5% NH₄HCO₃ and CH₃CN (40% CH₃CN up to 60% in 7 min, hold 95% in 1 min, down to 40% in 1 min); Detector, uv 254 nm. This resulted in 11.4 mg (8%) of (2E)-N,N-dimethyl-4-[[2-([5-[(1E)-4,4,4-trifluoro-1-(1H-indol-5-yl)-1-phenylbut-1-en-2-yl]pyridin-2-yl]oxy)ethyl]amino]but-2-enamide as a off-white solid. ¹H NMR (300 MHz, Methanol-d₄) δ ppm: 7.64 (d, J=2.4 Hz, 1H), 7.52 (d, J=1.5 Hz, 1H), 7.46-7.42 (m, 1H), 7.31 (d, J=3.3 Hz, 1H), 7.27-7.15 (m, 6H), 6.98-6.94 (m, 1H), 6.80-6.76 (m, 1H), 6.62-6.46 (m, 3H), 4.27 (t. J=5.4 Hz, 2H), 3.51-3.39 (m, 4H), 3.09 (s, 3H), 2.98 (s, 3H), 2.97-2.88 (m, 2H) ppm. LCMS: 548.6 [M+H]⁺.

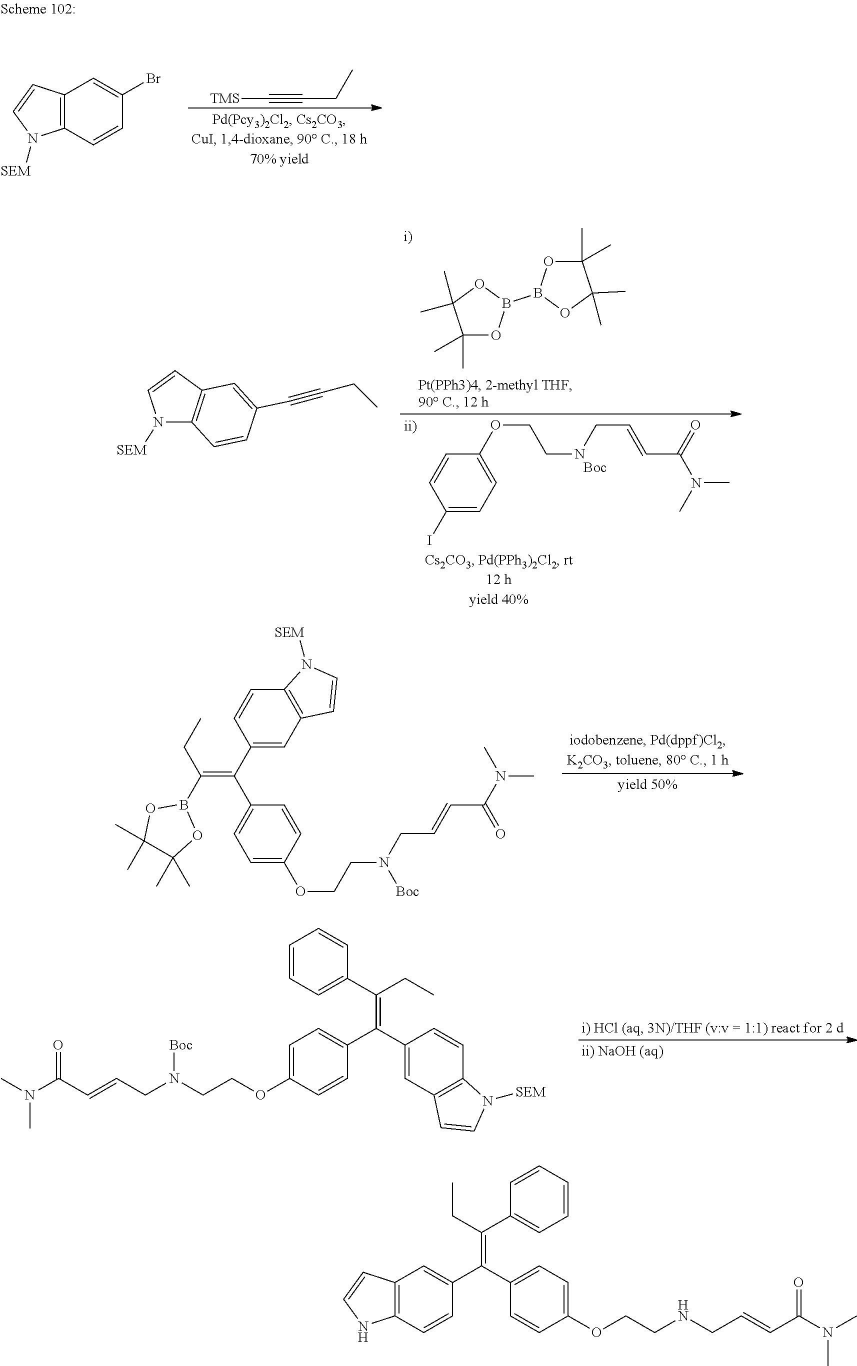

### Example 102: Synthesis of (E)-4-(2-(4-((E)-1-(1H-indol-5-yl)-2-phenylbut-1-enyl)phenoxy)ethyl amino)-N,N-dimethylbut-2-enamide (Compound 102)

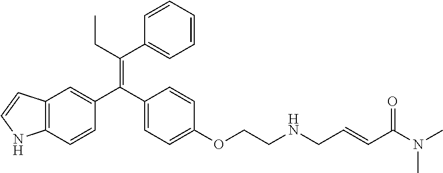 [SMILES: CC/C(=C(/c1ccc(OCCNC/C=C/C(=O)N(C)C)cc1)c1ccc2[nH]ccc2c1)c1ccccc1]

### Step-1: Synthesis of 5-(but-1-ynyl)-1-((2-(trimethylsilyl)ethoxy)methyl)-1H-indole

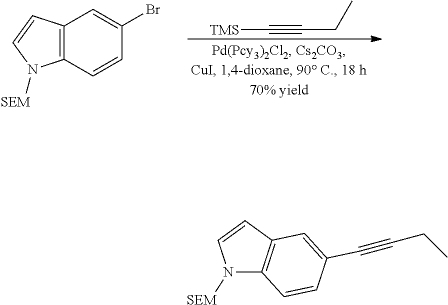

Into a 20-mL sealed tube purged and maintained with an inert atmosphere of nitrogen, was placed 5-bromo-1-[[2-(trimethylsilyl)ethoxy]methyl]-1H-indole (1 g, 3.06 mmol, 1.00 equiv), dioxane (5 mL), but-1-yn-1-yltrimethylsilane (3.8 g, 30.09 mmol, 10.00 equiv), Cs₂CO₃ (2 g, 6.14 mmol, 2.00 equiv), CuI (290 mg, 1.52 mmol, 0.05 equiv), Pd(PCy₃)₂Cl₂ (0.227 g, 0.10 equiv). The resulting solution was stirred for 18 h at 90° C. in an oil bath. The reaction mixture was cooled to 0° C. then quenched with ice cold water (10 mL) and extracted with dichloromethane (25 mL*3). The combined organic extracts were washed with brine (25 mL), dried over anhydrous sodium sulfate and concentrated under reduced pressure. The resulting mixture was concentrated under vacuum. The residue was applied onto a silica gel column with ethyl acetate/petroleum ether (1:10). This resulted in 700 mg (70%) of 5-(but-1-yn-1-yl)-1-[[2-(trimethylsilyl)ethoxy]methyl]-1H-indole as yellow oil.

### Step-2: Synthesis of Tert-butyl (E)-4-(dimethylamino)-4-oxobut-2-enyl(2-(4-((Z)-2-(4,4,5,5-tetramethyl-1,3,2-dioxaborolan-2-yl)-1-(1-((2-(trimethylsilyl)ethoxy)methyl)-1H-indol-5-yl)but-1-enyl)phenoxy)ethyl)carbamate

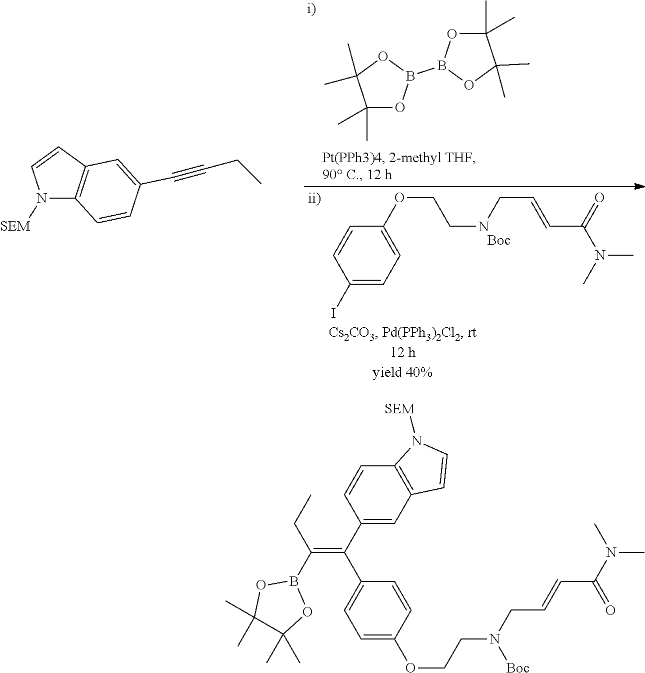

Into a 40-mL vial, was placed 5-(but-1-yn-1-yl)-1-[[2-(trimethylsilyl)ethoxy]methyl]-1H-indole (660 mg, 2.20 mmol, 1.00 equiv), 4,4,5,5-tetramethyl-2-(tetramethyl-1,3, 2-dioxaborolan-2-yl)-1,3,2-dioxaborolane (564 mg, 2.22 mmol, 1.00 equiv), Pt(pph₃)₄ (28 mg, 0.10 equiv), 2-methyl-THF (20 mL). This mixture was degassed and maintained with N₂. The resulting solution was stirred for 12 h at 90° C. in an oil bath. The solution was allowed to cool to room temperature, and (E)-tert-butyl 4-(dimethylamino)-4-oxobut-2-enyl(2-(4-iodophenoxy)ethyl)carbamate (1.05 g, 2.21 mmol, 1.00 equiv), bis(triphenylphosphine)palladium (II) dichloride (78 mg, 0.11 mmol, 0.05 equiv), cesium carbonate (1.44 g, 4.42 mmol, 2.00 equiv) and 2-methyl THF (20 mL), water (4 mL) were added. This mixture was stirred at room temperature for 12 h. The reaction mixture was quenched with water (20 mL) and extracted with dichloromethane (2×50 mL). The combined organic extracts were washed with brine (50 mL), dried over sodium sulfate and concentrated under vacuum. The crude material was purified by column chromatography over 100-200 mesh silica using 20% ethyl acetate in petrol ether to afford This resulted in 700 mg (42%) of tert-butyl N-[(2E)-3-(dimethylcarbamoyl)prop-2-en-1-yl]-N-(2-[4-[(1Z)-2-(tetramethyl-1,3,2-dioxaborolan-2-yl)-1-(1-[[2-(trimethylsilyl)ethoxy]methyl]-1H-indol-5-yl)but-1-en-1-yl]phenoxy]ethyl)carbamate as yellow oil.

### Step-3: Synthesis of Tert-butyl (E)-4-(dimethylamino)-4-oxobut-2-enyl(2-(4-((E)-2-phenyl-1-(1-((2-(trimethylsilyl)ethoxy)methyl)-1H-indol-5-yl)but-1-enyl)phenoxy)ethyl)carbamate

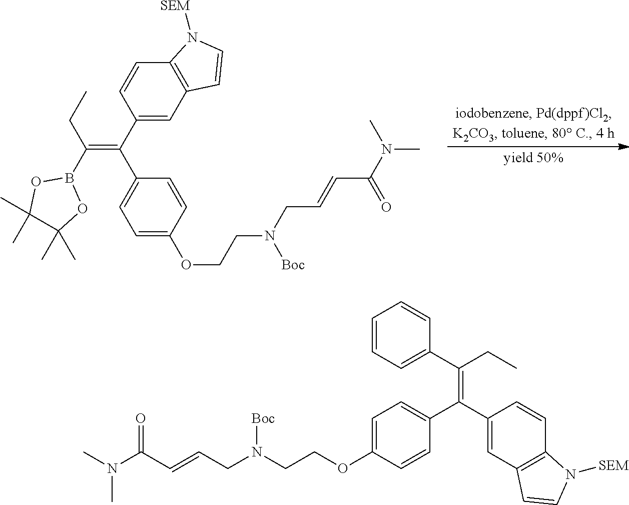

Into a 40-mL round-bottom flask, was placed tert-butyl N-[(2E)-3-(dimethylcarbamoyl)prop-2-en-1-yl]-N-(2-[4-[(1Z)-2-(tetramethyl-1,3,2-dioxaborolan-2-yl)-1-(1-[[2-(trimethylsilyl)ethoxy]methyl]-1H-indol-5-yl)but-1-en-1-yl]phenoxy]ethyl)carbamate (490 mg, 0.63 mmol, 1.00 equiv), iodobenzene (258 mg, 1.26 mmol, 2.00 equiv), potassium carbonate (175 mg, 1.27 mmol, 2.00 equiv), Pd(dppf)Cl2 (47 mg, 0.06 mmol, 0.10 equiv), water (2 mL), toluene (10 mL). This mixture was degassed and maintained with N₂. The resulting solution was stirred for 4 h at 80° C. in an oil bath. The reaction mixture was quenched with water (20 mL) and extracted with dichloromethane (2×50 mL). The combined organic extracts were washed with brine (50 mL), dried over sodium sulfate and concentrated under vacuum. The residue was applied onto a silica gel column with ethyl acetate/petroleum ether (1:10). This resulted in 250 mg (55%) of tert-butyl N-[(2E)-3-(dimethylcarbamoyl)prop-2-en-1-yl]-N-(2-[4-[(1E)-2-phenyl-1-(1-[[2-(trimethylsilyl)ethoxy]methyl]-1H-indol-5-yl)but-1-en-1-yl]phenoxy]ethyl)carbamate as yellow oil.

### Step-4: Synthesis of (E)-4-(2-(4-((E)-1-(1H-Indol-5-yl)-2-phenylbut-1-enyl) phenoxy)ethylamino)-N,N-dimethylbut-2-enamide

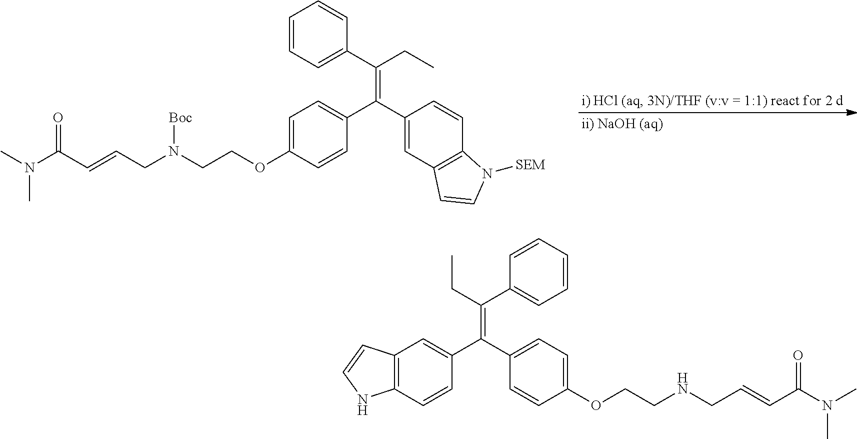 [SMILES: CC/C(=C(/c1ccc(OCCN(C/C=C/C(=O)N(C)C)C(=O)OC(C)(C)C)cc1)c1ccc2c(ccn2COCC[Si](C)(C)C)c1)c1ccccc1 ; CC/C(=C(/c1ccc(OCCNC/C=C/C(=O)N(C)C)cc1)c1ccc2[nH]ccc2c1)c1ccccc1]

Into a 500-mL round-bottom flask, was placed tert-butyl N-[(2E)-3-(dimethylcarbamoyl)prop-2-en-1-yl]-N-(2-[4-[(1E)-2-phenyl-1-(1-[[2-(trimethylsilyl)ethoxy]methyl]-1H-indol-5-yl)but-1-en-1-yl]phenoxy]ethyl)carbamate (250 mg, 0.35 mmol, 1.00 equiv), hydrochloric acid (3N)/THF (v/v=1:1, 300 mL). The resulting solution was stirred for 48 h at 0° C. The resulting solution was extracted with 2×100 mL of ethyl acetate and the organic layers combined. The crude product was purified by Prep-HPLC with the following conditions (2#-AnalyseHPLC-SHIMADZU(HPLC-10)): Column. XBridge Shield RP18 OBD Column: 5 um, 19×150 mm; mobile phase, water (0.05% NH3H2O) and ACN (30.0% ACN up to 65.0% in 15 min); Detector, UV 220 nm. This resulted in 20.6 mg (12%) of (2E)-4-[(2-[4-[(1E)-1-(1H-indol-5-yl)-2-phenylbut-1-en-1-yl]phenoxy]ethyl)amino]-N,N-dimethylbut-2-enamide as a pink solid. 1H NMR (300 MHz, Methanol-d4) δ 7.42 (s, 1H), 7.35 (d, J=8.4 Hz, 1H), 7.24 (d, J=3.1 Hz, 1H), 7.17-7.13 (m, 5H), 6.93 (d, J=8.4 Hz, 1H), 6.83-6.76 (m, 3H), 6.61-6.56 (m, 3H), 6.43 (dd, J=3.2, 0.9 Hz, 1H), 4.07 (t. J=5.2 Hz, 2H), 3.52 (dd, J=5.6, 1.6 Hz, 2H), 3.16 (s, 3H), 3.02-2.93 (m, 5H), 2.52 (m, 2H), 0.96 (t, J=7.4 Hz, 3H) ppm. LCMS: 494.0 [M+H]⁺.

### Example 103: Preparation of Substituted Analogs

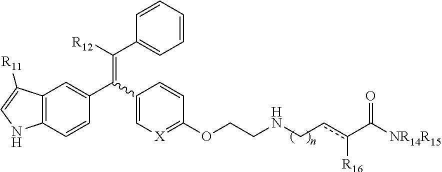

Compounds with the above general formula may be prepared by following a similar reaction sequence to that described in scheme 1, wherein the R₁₂ substituent is introduced by using the appropriately substituted iodide in place of 1,1,1-trifluoro-2-iodoethane in step 4.

The R₁₄, R₁₅, and R₁₆ substituents are introduced, and the value of “n” and status of [symbol] as a double or single bond are determined, by using an appropriately substituted phenyl (X═C) or pyridyl (X═N) iodide in step 6, for example:

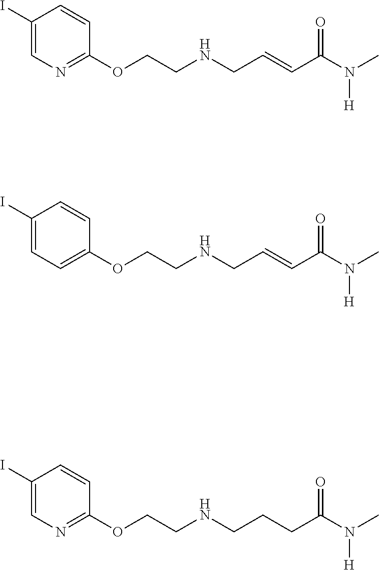 [SMILES: CNC(=O)/C=C/CNCCOc1ccc(I)cn1 ; CNC(=O)/C=C/CNCCOc1ccc(I)cc1 ; CNC(=O)CCCNCCOc1ccc(I)cn1]

The R₁₁ substituent can be varied by selection of the appropriately substituted starting material, for example:

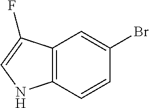 [SMILES: Fc1c[nH]c2ccc(Br)cc12]

The R₁₂ substituent can also be introduced by selection of the appropriately substituted TMS-acetylene in place of but-1-yn-1-yltrimethylsilane in step 1 of scheme 2.

### Example 201—Compounds that Inhibit ERα^{WT/MUT} Activity In Vitro

### Cell Culture

MCF7 BUS cells (Coser, et al., (2003) PNAS 100(24): 13994-13999) were maintained in Dulbecco's Modified Eagle Medium supplemented with 10%/o FBS, 4 mM L-glutamine and 1× non-essential amino acids. Lenti-X 293T cells (Clontech, Cat #632180) were routinely cultured in Dulbecco's Modified Eagle Medium supplemented with 10% FBS.

### Site-Direct Mutagenesis and Cell Line Engineering

The QuikChange II XL Site-Directed Mutagenesis Kit (Agilent Technologies, Cat #200523) was used to generate Y537S, Y537C, Y537N and D538G mutations within the ERα exon 8. Wild-type ESR1 cDNA (GeneCopoeia Inc., Cat# GC-A0322, accession no. NM 000125) was used as a template with the following mutagenesis primers (where the underlined nucleotides represent site mutations); Y537S: F-AAG AAC GTG GTG CCC CTC TCT GAC CTG CTG CTG GAG ATG (SEQ ID NO: 1), R-CAT CTC CAG CAG CAG GTC AGA GAG GGG CAC CAC GTT CTT (SEQ ID NO: 2): Y537N: F-AAG AAC GTG GTG CCC CTC AAT GAC CTG CTG CTG GAG ATG (SEQ ID NO: 3), R-CAT CTC CAG CAG CAG GTC ATT GAG GGG CAC CAC GTT CTT (SEQ ID NO: 4); Y537C: F-AAG AAC GTG GTG CCC CTC TGT GAC CTG CTG CTG GAG ATG (SEQ ID NO: 5), R-CAT CTC CAG CAG CAG GTC ACA GAG GGG CAC CAC GTT CTT (SEQ ID NO: 6): D538G: F-AAC GTG GTG CCC CTC TAT GGC CTG CTG CTG GAG ATG CTG (SEQ ID NO: 7), R-CAG CAT CTC CAG CAG CAG GCC ATA GAG GGG CAC CAC GTT (SEQ ID NO: 8). WT and mutant ESR1 cDNAs were cloned into the designation lentiviral vector pLenti6.3N/V5-Dest (Invitrogen, Cat #V533-06). To make lentivirus, DNAs (WT and mutant ESR1) were co-transfected with packaging plasmids into Lenti-X 293T cells using TransIT (Mirus, Cat #MIR 2700). 48 h post-transfection, virus containing media was filtered and added to MCF7 cells in the presence of 8 μg/ml polybrene overnight. Two days following infection, cells were placed under selection with 10 μg/ml blasticidin for 2 weeks for stable expression.

### In Vitro Proliferation Assays

MCF7-WT and —Y537S cells were seeded at 1500 cells/well in black-walled 96-well plates (assay plates. Costar, Cat #3904). In parallel, cells were also seeded in a separate 96-well plate (8 wells/cell line, control plate) for which a CTG (CellTiter-Glo® Luminescent Viability Assay, Promega, Cat #G7572) was measured the following day (day 0 reading). The day 0 reading was used for the GI₅₀ calculation at the termination of the experiment. The day following seeding, compounds were added to assay plates. Briefly, a 1:4 serial dilution was prepared in DMSO at 200× final concentration for a total of 10 concentrations (9 dilutions containing compound and one is DMSO only). Serially diluted compounds were pipetted into medium to prepare a compound-medium mix at 10× final concentration. 10 μl of compound-medium mix was added to MCF7-WT and —Y537S cells at 3 wells/concentration (triplicate for each concentration). On day 3, media-compound was removed and replaced with fresh media/compound as described above. On day 6. CTG was measured and compared to day 0 readings from control plate to assess GI₅₀.

### Results

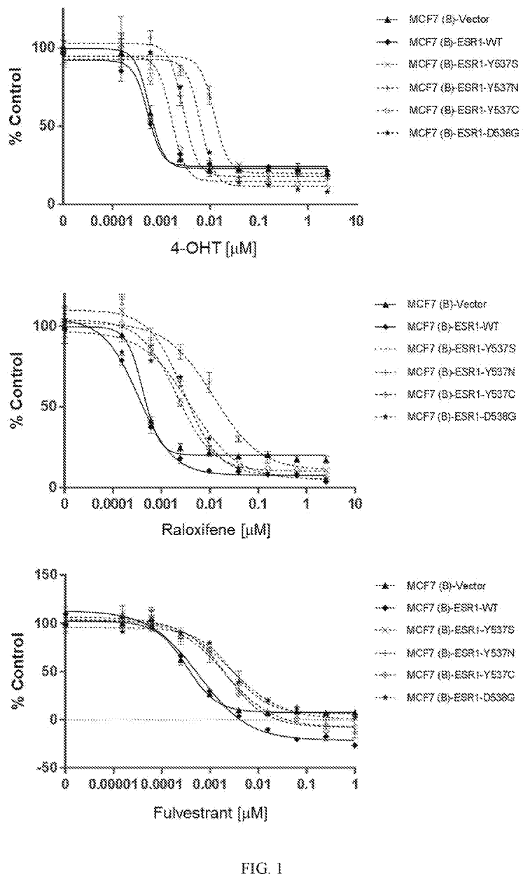

FIG. 1 shows that ectopic expression of ERα^{Y537S/N/C, D538G} in MCF7 cells conferred phenotypic resistance to currently marketed therapies tamoxifen (SERM), raloxifene (SERM) and fulvestrant (SERD). Similar observations were also recently published by several independent labs (Jeselsohn et al., (2014) Clin Cancer Res. April 1; 20(7): 1757-67; Toy et al., (2013) Nat Genet. 2013 December; 45(12): 1439-45; Robinson et al., (2013) Nat Genet. December; 45(12):1446-51; Merenbakh-Lamin et al., (2013) Cancer Res. December 1; 73(23):6856-64; Yu et al., (2014) Science July 11; 345(6193):216-20). Having confirmed that ERα^{MUT} drive resistance to current endocrine therapies, identification of novel compounds that would reduce proliferation of the ERα^{MUT}-bearing MCF7 cells more efficaciously than the corresponding clinical compound 4-hydroxytamoxifen was sought. Using the WT and mutant viability assay as a screening tool, compounds were identified that were more potent towards the Y537S-bearing MCF7 line relative to 4-hydroxytamoxifen. The results of the viability assay screen are shown in Table 2 above.

### In Vivo Xenograft Methods

### Methods and Materials

Although not wishing to be bound by theory, applicant appreciates that certain in vivo xenograft studies may be useful in identifying effective compounds. Such studies may be conducted, for example, using compounds reported herein and/or their salts. In some embodiments their hydrochloride salts are used in the studies. The MCF7 xenograft study, Y537S positive PDx xenograft study, and WHIM20 xenograft study reported below have not yet been conducted with compounds reported herein.

### MCF7 Xenograft Study

The ESR1 wild-type human ER+ breast cancer cell line MCF7 (ATCC) is cultured in DMEM media supplemented with 10% FBS at 37° C. in a 5% CO2 atmosphere and kept in the exponential growth phase. The cells are collected in trypsin and re-suspended in a 1:1 mixture of matrigel and HBSS at a final concentration of 5×10⁷ cells/mL. A 0.2 mL aliquot of cells is injected subcutaneously into the 3^{rd} mammary fat pad of 6-8 week old female Balb/c nude mice, giving 1×10⁷ cells/mouse. When the average tumor volume reaches approximately about 200 mm³, animals are randomized prior to treatment.

All of the compounds are dosed orally every day at doses ranging from 1 to 30 mg/kg. Each treatment is started on Day 0 and the administration schedule is continued for 17 days. The administration volume is calculated from the individual mouse body weights prior to dose administration. The body weights are measured daily while the tumor volumes are measured twice a week. Tumor volumes (TV) are calculated based on the formula:

TV=length×width²×0.5

length: largest diameter of tumor (mm)
width: diameter perpendicular to length (mm)
The Tumor Growth Inhibition % (TGI) is calculated according to the following formula:

$$Tumor Growth Inhibition \% ( T G I ) = \frac{\begin{matrix}\text{Average Control TV Day X-} \\ \text{Treatment TV Day X}\end{matrix}}{\text{Average Control TV Day X}} \times 100$$

Where Day X is the endpoint measurement.

### Y537S Positive PDx Xenograft Study

A Patient-Derived Xenograft (PDX) tumor model representing an ESR1-Y537S mutated human ER+ breast cancer, designated as PDX-Y537S, is propagated subcutaneously in immunocompromised mice. The tumors are excised within 60 days of implantation and processed to mixed tumor fragments. Solid tumor tissues are depleted of necrotic components, cut into 70 mg fragments, mixed with matrigel and subcutaneously implanted into the right flank of 6-12 week old female athymic Nude (Crl:NU(NCr)-Foxn1nu) mice. The precise number of fragments and volume of matrigel are determined on a case by case basis. When the average tumor volume reaches approximately 200 mm³, animals are randomized prior to treatment. All of the primary human tumors utilized in this study undergo approximately 7 passages in vivo.

Estrogen is not supplemented in the studies. All tested compounds are dosed orally every day at doses ranging from 3 to 200 mg/kg. Each treatment is started on Day 0 and the administration schedule is continued for up to 35 days. The administration volume is calculated from the individual mouse body weights prior to dose administration. The body weights are measured daily while the tumor volumes are measured twice a week. Tumor volumes are calculated based on the previously described formula.

### WHIM20 Xenograft Study

The Patient-Derived Xenograft (PDX) tumor model, WHIM20, representing an ESR1-Y537S mutated human ER+ breast cancer is propagated in mice. The tumors are excised and processed to mixed tumor fragments and the fragments are re-implanted subcutaneously into new recipient mice. Solid tumor tissues are depleted of necrotic components, cut into fragments, mixed with matrigel and subcutaneously implanted into the right flank of 6-8 week old female SCID-bg mice. The precise number of fragments and volume of matrigel are determined on a case by case basis. When the average tumor volume reaches approximately 200 mm³, animals are randomized prior to treatment. All of the primary human tumors utilized in this study undergo approximately 4 passages in vivo.

Estrogen is not supplemented in WHIM20 studies. Compounds are dosed orally every day at the indicated doses. Each treatment is started on Day 0 and the administration schedule is continued for the indicated days. The administration volume is calculated from the individual mouse body weights prior to dose administration. The body weights are measured daily while the tumor volumes are measured twice a week. Tumor volumes are calculated based on the previously described formula.

### Statistical Analysis

Data are expressed as the mean±SEM for tumor volume and the mean±SEM for body weight. The differences in tumor volume during the study period between the vehicle treated and compound treated groups are analyzed by two-way analysis of variance (ANOVA) followed by the Dunnett multiple comparison post hoc test. Statistical analyses are performed using the GraphPad Prism® version 5.04 (GraphPad Software, La Jolla, Calif.).

It will now be apparent that new, improved, and nonobvious compositions have been described in this specification with sufficient particularity as to be understood by one of ordinary skill in the art. Moreover, it will be apparent to those skilled in the art that modifications, variations, substitutions, and equivalents exist for features of the compositions which do not materially depart from the spirit and scope of the embodiments disclosed herein. Accordingly, it is expressly intended that all such modifications, variations, substitutions, and equivalents which fall within the spirit and scope of the invention as defined by the appended claims shall be embraced by the appended claims.

1. (canceled)

2. A compound of Formula (X): 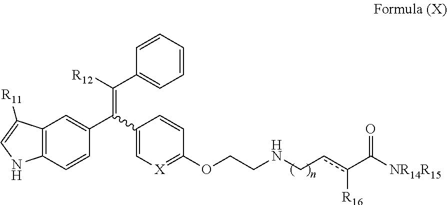
wherein:
R₁₁ is —H or —F;
R₁₂ is —CH₂CH₃, —CH₂CF₃, or cyclobutyl;
R₁₄ and R₁₅
i) are the same or different and are independently selected from —H, —CH₃, and —CH₂CH₂OH; or
ii) form a 4-6 membered heterocycloalkyl ring with the N to which they are attached, optionally with an additional heteroatom in the 4-6 membered ring;
R₁₆ is —H or forms a 5-7 membered heterocycloalkyl ring with R₁₄ and the N to which R₁₄ is attached;
X is N or C;
n is 1-2; [symbol]
represents a single bond or a double bond;
wherein R₁₆ is H when R₁₄ and R₁₅ form said 4-6 membered heterocycloalkyl ring; and
wherein R₁₅ is selected from —H, —CH₃, and —CH₂CH₂OH when R₁₆ forms said 5-7 membered heterocycloalkyl ring with R₁₄;
or a pharmaceutically acceptable salt thereof.

3. The compound or pharmaceutically acceptable salt of claim 2, having the stereochemistry set forth in Formula (XI): 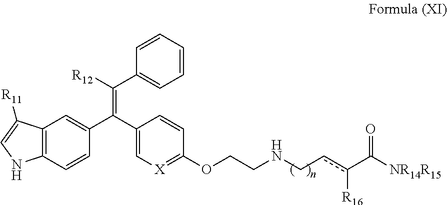
or a pharmaceutically acceptable salt thereof.

4. The compound or pharmaceutically acceptable salt of claim 2, wherein R₁₁ is —F.

5. The compound or pharmaceutically acceptable salt of claim 2, wherein R₁₁ is —H.

6. The compound or pharmaceutically acceptable salt of claim 2, wherein R₁₂ is —CH₂—CF₃.

7. The compound or pharmaceutically acceptable salt of claim 2, wherein R₁₂ is —CH₂CH₃.

8. The compound or pharmaceutically acceptable salt of claim 2, wherein X is N.

9. The compound or pharmaceutically acceptable salt of claim 2, wherein R₁₄ is H and R₁₅ is —CH₃.

10. The compound or pharmaceutically acceptable salt of claim 2, wherein R₁₄ and R₁₅ are —CH₃.

11. The compound or pharmaceutically acceptable salt of claim 2, wherein [symbol] represents a double bond.

12. A compound or pharmaceutically acceptable salt selected from the group consisting of:
(2E)-N,N-dimethyl-4-[[2-([5-[(1E)-4,4,4-trifluoro-1-(1H-indol-5-yl)-1-phenylbut-1-en-2-yl]pyridin-2-yl]oxy)ethyl]amino]but-2-enamide;
(E)-4-(2-(4-((E)-1-(1H-indol-5-yl)-2-phenylbut-1-enyl)phenoxy)ethyl amino)-N,N-dimethylbut-2-enamide;
(E)-N,N-dimethyl-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-enamide;
(E)-N-methyl-4-((2-((5-((Z)-4,4,4-trifluoro-1-(1H-indol-5-yl)-2-phenylbut-1-en-1-yl)pyridin-2-yl)oxy)ethyl)amino)but-2-enamide;
(E)-N-methyl-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-enamide;
(E)-N-methyl-4-((2-(4-((E)-4,4,4-trifluoro-1-(1H-indol-5-yl)-2-phenylbut-1-en-1-yl)phenoxy)ethyl)amino)but-2-enamide;
(E)-N-methyl-4-((2-(4-((E)-4,4,4-trifluoro-1-(3-fluoro-1H-indol-5-yl)-2-phenylbut-1-en-1-yl)phenoxy)ethyl)amino)but-2-enamide;
(E)-1-(pyrrolidin-1-yl)-4-((2-((5-((Z)-4,4,4-trifluoro-1-(1H-indol-5-yl)-2-phenylbut-1-en-1-yl)pyridin-2-yl)oxy)ethyl)amino)but-2-en-1-one;
(E)-1-(pyrrolidin-1-yl)-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-en-1-one;
(E)-4-((2((5-((Z)-2-cyclobutyl-1-(1H-indol-5-yl)-2-phenylvinyl)pyridin-2-yl)oxy)ethyl)amino)-N-methylbut-2-enamide;
(E)-N-(2-hydroxyethyl)-4-((2-((5-((Z)-4,4,4-trifluoro-1-(3-fluoro-1H-indol-5-yl)-2-phenylbut-1-en-1-yl)pyridin-2-yl)oxy)ethyl)amino)but-2-enamide;
(E)-N-(2-hydroxyethyl)-4-((2((5-((Z)-4,4,4-trifluoro-1-(1H-indol-5-yl)-2-phenylbut-1-en-1-yl)pyridin-2-yl)oxy)ethyl)amino)but-2-enamide;
(E)-N-(2-hydroxyethyl)-5-((2-((5-((Z)-4,4,4-trifluoro-1-(3-fluoro-1H-indol-5-yl)-2-phenylbut-1-en-1-yl)pyridin-2-yl)oxy)ethyl)amino)pent-2-enamide;
(Z)—N-methyl-4-((2-((5-(4,4,4-trifluoro-1-(3-fluoro-1H-indol-5-yl)-2-phenylbut-1-en-1-yl)pyridin-2-yl)oxy)ethyl)amino)butanamide;
(Z)—N-methyl-4-((2-((5-(4,4,4-trifluoro-1-(1H-indol-5-yl)-2-phenylbut-1-en-1-yl)pyridin-2-yl)oxy)ethyl)amino)butanamide;
(Z)—N-methyl-5-((2-((5-(4,4,4-trifluoro-1-(3-fluoro-1H-indol-5-yl)-2-phenylbut-1-en-1-yl)pyridin-2-yl)oxy)ethyl)amino)pentanamide;
(Z)—N-methyl-5-((2-((5-(4,4,4-trifluoro-1-(1H-indol-5-yl)-2-phenylbut-1-en-1-yl)pyridin-2-yl)oxy)ethyl)amino)pentanamide;
(Z)-3-(2-((2-((5-(4,4,4-trifluoro-1-(3-fluoro-1H-indol-5-yl)-2-phenylbut-1-en-1-yl)pyridin-2-yl)oxy)ethyl)amino)ethyl)pyrrolidin-2-one;
(Z)-3-(2-((2-((5-(4,4,4-trifluoro-1-(1H-indol-5-yl)-2-phenylbut-1-en-1-yl)pyridin-2-yl)oxy)ethyl)amino)ethyl)pyrrolidin-2-one;
(E)-4-((2-((5-((Z)-1-(3-fluoro-1H-indol-5-yl)-2-phenylbut-1-en-1-yl)pyridin-2-yl)oxy)ethyl)amino)-N-methylbut-2-enamide;
(E)-4-((2-(4-((E)-1-(3-fluoro-1H-indol-5-yl)-2-phenylbut-1-en-1-yl)phenoxy)ethyl)amino)-N-methylbut-2-enamide;
(E)-4-((2-(4-((E)-2-cyclobutyl-1-(3-fluoro-1H-indol-5-yl)-2-phenylvinyl)phenoxy)ethyl)amino)-N-methylbut-2-enamide;
(E)-4-((2-((5-((Z)-2-cyclobutyl-1-(3-fluoro-1H-indol-5-yl)-2-phenylvinyl)pyridin-2-yl)oxy)ethyl)amino)-N-methylbut-2-enamide; and
(E)-4-((2-((5-((Z)-2-cyclobutyl-1-(1H-indol-5-yl)-2-phenylvinyl)pyridin-2-yl)oxy)ethyl)amino)-N-methylbut-2-enamide; or a pharmaceutically acceptable salt thereof.

13. The compound of claim 2, with the following formula: 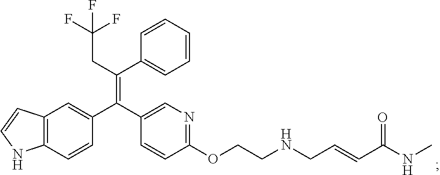 [SMILES: CNC(=O)/C=C/CNCCOc1ccc(/C(=C(/CC(F)(F)F)c2ccccc2)c2ccc3[nH]ccc3c2)cn1]
or a pharmaceutically acceptable salt thereof.

14. The compound of claim 2, with the following formula: 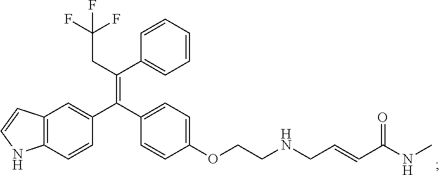 [SMILES: CNC(=O)/C=C/CNCCOc1ccc(/C(=C(/CC(F)(F)F)c2ccccc2)c2ccc3[nH]ccc3c2)cc1]
or a pharmaceutically acceptable salt thereof.

15. A compound of Formula (XII) or a pharmaceutically acceptable salt thereof: 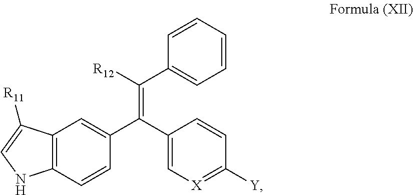
wherein R₁₁ is —H or —F;
R₁₂ is —CH₂CH₃, —CH₂CF₃, or cyclobutyl;
X is N or C; and
Y is one of the following:

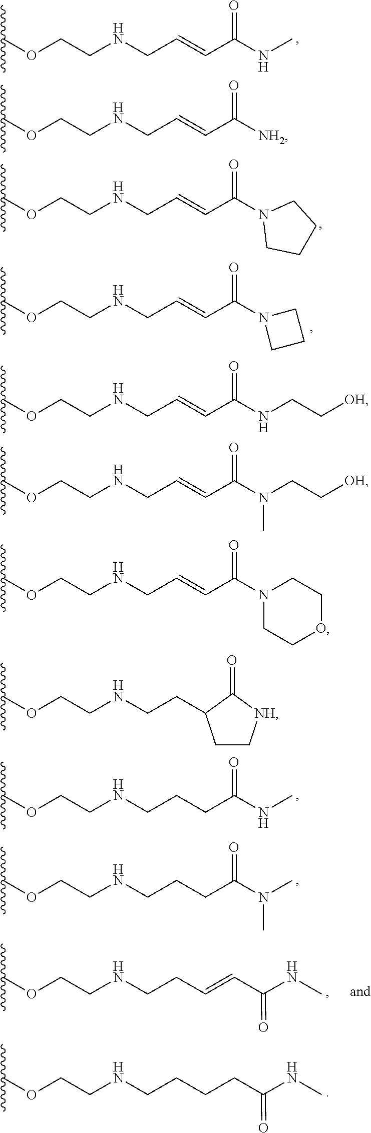

16. A pharmaceutical composition comprising a compound or pharmaceutically acceptable salt of claim 2 and a pharmaceutically acceptable carrier.

17. A method of treating breast cancer comprising administering to a subject in need of treatment an effective amount of the pharmaceutical composition of claim 16.

18. The method of claim 17, wherein the breast cancer is an ER-positive breast cancer.

19. The method of claim 17, wherein the subject expresses a mutant ER-α protein.

20. A pharmaceutical composition comprising a compound or pharmaceutically acceptable salt of claim 15 and a pharmaceutically acceptable carrier.

21. A method of treating breast cancer comprising administering to a subject in need of treatment an effective amount of the pharmaceutical composition of claim 20.


SEQUENCE LISTING (8 sequences)

SEQ ID NO: 1 - DNA, 39 bp, Artificial Sequence
  ESR Y537S Mutation Forward Primer
aagaacgtggtgcccctctctgacctgctgctggagatg

SEQ ID NO: 2 - DNA, 39 bp, Artificial Sequence
  ESR Y537S Mutation Reverse Primer
catctccagcagcaggtcagagaggggcaccacgttctt

SEQ ID NO: 3 - DNA, 36 bp, Artificial Sequence
  ESR Y537N Mutation Forward Primer
aagaacgtggtgcccctcaatgacctgctgctggag

SEQ ID NO: 4 - DNA, 36 bp, Artificial Sequence
  ESR Y537N Mutant Reverse Primer
catctccagcagcaggtcattgaggggcaccacgtt

SEQ ID NO: 5 - DNA, 39 bp, Artificial Sequence
  ESR Y537C mutant forward primer
aagaacgtggtgcccctctgtgacctgctgctggagatg

SEQ ID NO: 6 - DNA, 39 bp, Artificial Sequence
  ESR Y537C mutant reverse primer
catctccagcagcaggtcacagaggggcaccacgttctt

SEQ ID NO: 7 - DNA, 39 bp, Artificial Sequence
  ESR D538G mutant forward primer
aacgtggtgcccctctatggcctgctgctggagatgctg

SEQ ID NO: 8 - DNA, 39 bp, Artificial Sequence
  ESR D538G Mutant Reverse primer
cagcatctccagcagcaggccatagaggggcaccacgtt